(19) United States
(12) Patent Application Publication
(10) Pub. No.: US 2017/0335290 A1
(43) Pub. Date: Nov. 23, 2017

(54) SURVIVIN SPECIFIC T-CELL RECEPTOR TARGETING TUMOR BUT NOT T CELLS

(71) Applicant: Baylor College of Medicine, Houston, TX (US)

(72) Inventors: Barbara Savoldo, Chapel Hill, NC (US); Gianpietro Dotti, Chapel Hill, NC (US); Caroline Eva Arber Barth, Bellaire, TX (US)

(21) Appl. No.: 15/522,698

(22) PCT Filed: Oct. 30, 2015
(86) PCT No.: PCT/US15/58452

§ 371 Date: Apr. 27, 2017

Related U.S. Application Data
(60) Provisional application No. 62/073,076, filed on Oct. 31, 2014.

(51) Int. Cl. C12N 5/09; C07K 14/705; C12N 5/0783

(52) U.S. Cl. CPC C12N 5/0694; C12N 5/0636; C07K 14/705; C12N 2510/00

(57) ABSTRACT

Embodiments of the disclosure concern engineered T cell receptors that are specific for the survivin tumor antigen but do not have “on-target off tumor” toxicity. In particular embodiments, particular alpha and beta chains are utilized in engineered T cell receptors for cell therapy that have effective anti-tumor activity but lack fratricidal effects. Methods, compositions, and kits are provided herein.

This application claims priority to U.S. Provisional Patent Application Ser. No. 62/073,076, filed Oct. 31, 2014, which application is incorporated by reference herein in its entirety.

### STATEMENT REGARDING FEDERALLY SPONSORED RESEARCH OR DEVELOPMENT

This invention was made with government support under R01 CA131027 and P50 CA126752 awarded by the National Cancer Institute. The government has certain rights in the invention.

### TECHNICAL FIELD

Embodiments of the disclosure concern at least the fields of immunology, cell biology, molecular biology, and medicine, including cancer medicine.

### BACKGROUND OF THE INVENTION

Cancer-targeted adoptive T-cell therapy with genetically engineered αβT-cell receptors (TCRs) has resulted in encouraging responses in some patients (Morgan, et al., 2006; Johnson, et al., 2009; Robbins, et al., 2011). Broadening this approach to a larger array of malignancies requires targeting more widely expressed tumor-associated antigens (TAAs). However, most TAAs are not exclusively tumor-specific but are also expressed at low levels in normal adult tissues, making TCR-mediated targeting of these important antigens a challenge. “On-target off-tumor” toxicity may occur when TCRs fail to discriminate levels of TAA presented on normal versus tumor cells, for example when the antigen is expressed equally, or when the TCR not only recognizes low levels of the targeted TAA-epitope but also a cross-reactive epitope expressed on normal cells. Such combined target recognition may then lead to T cell activation resulting in toxicity that apparently precludes safe targeting of the desired TAA. To characterize this putative mechanism the TAA survivin was used as a model. Survivin was prioritized as a target by the National Cancer Institute for the development of immunotherapies (Chever, et al., 2009) because of its ubiquitous over-expression in cancer and its crucial role in maintaining tumor cell phenotype and functions. Furthermore, compelling results from previous studies suggested that it is an excellent model antigen to study the problem of antigen threshold sensing and molecular discrimination. Autologous vaccination with survivin-derived peptides has proven safe (Rapoport, et al., 2011) and effective in inducing survivin-specific T-cell precursors (Becker, et al., 2012), but objective clinical responses remain limited (Becker, et al., 2012). Conversely, T cells expressing transgenic survivin-specific TCRs isolated from allo-restricted TCR repertoires circumventing thymic selection have not only produced antitumor activity but also severe “fratricidal” effects or toxicity against activated T cells and were thus incapable of discriminating self from tumor (Leisegang, et al., 2010). This cytotoxic effect was considered “on-target off-tumor” as survivin mRNA was found up-regulated in activated T lymphocytes (Leisegang, et al., 2010).

It was considered, however, that selection from an autologous TCR repertoire should be able to identify survivin-specific clones with high affinity and selectivity capable of self versus tumor discrimination since highly auto/cross-reactive T-cell clones have already undergone thymic selection and surviving T cells should express TCRs “tolerant” to antigen thresholds present in healthy cells and tissues. This strategy is in sharp contrast to other TCR engineering approaches that aim at priming T-cell responses from allogeneic or xenogenic repertoires devoid of human thymic selection (Spranger, et al., 2012) or ex vivo generation of TCRs with high or supraphysiologic avidities (Li, et al., 2005). However, these methods have produced severe toxicities due to unrecognized cross-reactivities targeting epitopes from entirely unrelated proteins that can be expressed by healthy tissues (Cameron, et al., 2013; Linette, et al., 2013). Described herein is the successful cloning of a survivin-specific TCR from autologous cultures that has antitumor activity, but lacks “fratricidal” effects or toxicity against normal hematopoietic stem/progenitor cells. To understand the mechanistic basis of this striking difference in molecular recognition of TCRs isolated from autologous versus allogeneic TCR repertoires, alanine-substitution analysis of the survivin TCRs was performed. The observation was validated on a set of additional TCRs targeting other TAAs. These studies provide critical insights into the determinants governing selective TCR molecular recognition.

The present disclosure satisfies a need in the art by providing survivin-specific immunotherapies for cancer that lack toxicity to other cells, including non-cancer cells that express survivin.

### BRIEF SUMMARY

Embodiments of the disclosure encompass methods and compositions for cell therapy. In particular embodiments, the cell therapy comprises modified cells having a particular receptor. In specific embodiments, the cells are immune cells, including immune cells that are T cells that have a particular receptor comprising a particular amino acid sequence therein. In certain aspects, there are compositions having cells with a modified T cell receptor comprising particular alpha and beta chains that provide selective antitumor activity. In specific embodiments, the cells comprise one or both of an alpha chain of said receptor comprising SEQ ID NO:1 or a functional fragment or functional derivative thereof; and a beta chain of said receptor comprising SEQ ID NO:2 or a functional fragment or functional derivative thereof

In a first aspect, provided herein are genetically engineered immune cells, e.g., T cells (T lymphocytes), natural killer (NK) cells or NKT cells, that are directed to survivin and are specific for “on target on tumor” selectivity. In a specific embodiment, the genetically engineered immune cells are T cells. In specific embodiments, the genetically engineered immune cells, e.g., T cells, comprise a receptor that is sensitive for level of surviving expression on cancer cells but not for level of expression of survivin on non-cancer cells (or at least at a reduced level for survivin on non-cancer cells compared to cancer cells). In specific embodiments, when the TCR binds to the surviving-MHC complex on the cancer cells, the immune cell kills the cancer cells. In certain embodiments, the immune cell, e.g., T cell, comprises a TCR comprising one or both of SEQ ID NO:1 and SEQ ID NO:2 or functional fragments or derivatives thereof. Cells of the disclosure that may be modified to target cancers expressing survivin include at least T-cells (which may be referred to as cytotoxic T lymphocytes (CTLs)), NK-cells, NKT-cells, or any other cellular elements with the capability of inducing an effector immune response. In particular cases the cells harbor a polynucleotide that encodes the survivin-specific TCR.

In another aspect, provided herein are any of the survivin-specific TCR polypeptides described herein. Also provided are polynucleotides encoding such TCRs. In embodiments of the invention, there is a polynucleotide comprising sequence that encodes a survivin-specific TCR and particularly one with specific alpha and/or beta chains. In embodiments of the invention, there is a polynucleotide comprising sequence that encodes a survivin-specific TCR.

Any polynucleotide of the disclosure may be comprised in an expression vector, including one that is a viral vector, such as a retroviral vector, lentiviral vector, adenoviral vector, or adeno-associated viral vector. In specific embodiments, the vector is a non-viral vector, including non-viral vector-mediated gene transfer, such as sleeping beauty or piggyback and mRNA electroporation. In embodiments of the disclosure, there is a cell, comprising at least one of any expression vector of the disclosure. The cell may be a eukaryotic or prokaryotic cell. The cell may be an immune system cell. The cell may be a T cell, NK cell, or NKT cell, for example.

In embodiments, the cancer may be of any kind and of any stage. The individual having cancer may be of any age or either gender. In specific embodiments, the individual is known to have cancer, is at risk for having cancer, or is suspected of having cancer. The cancer may be a primary or metastatic cancer, and the cancer may be refractory to treatment with other modalities, e.g., chemotherapy, radiation, or the like. In specific embodiments, the cancer is a hemotological cancer (cancer of the blood and blood-forming tissues (such as the bone marrow), including acute and chronic leukemia, Hodgkin's and non-Hodgkin's lymphoma, and multiple myeloma) or non-hematological cancers. In particular embodiments, the non-hematological cancer is of the brain, skin, lung, breast, prostate, colon, pancreas, thyroid, bone, kidney, spleen, liver, gall bladder, bladder, rectum, endometrium, ovary, testis, cervix, and so forth.

In certain embodiments of the disclosure, the disclosure concerns methods and compositions related to therapeutic cells, including therapeutic immune system cells such as tumor-specific cytotoxic T lymphocytes. The cells may comprise cellular elements with the capability of inducing an effector immune response. In certain aspects, the cells express at least one non-endogenous molecule that targets a particular tumor antigen, and in at least some cases, the molecule comprises a TCR.

In embodiments of the disclosure, there is a method of treating an individual for cancer, comprising the step of providing a therapeutically effective amount of a plurality of any of cells of the invention. The cancer may comprise one or more tumors.

In embodiments of the disclosure there is a kit comprising at least one polynucleotide of the invention, at least one expression vector of the invention, and/or at least one cell or cells of the invention.

Embodiments of the disclosure provide survivin-specific T-cell receptors targeting cancer but not targeting T cells. Disclosed herein are T-cell receptors that provide epitope specificity, antitumor activity for survivin-expressing cancers, and lack of autoreactivity.

In certain embodiments, there is a composition comprising an immune cell, said cell comprising an engineered survivin-specific T cell receptor, wherein the receptor comprises one or both of the following: an alpha chain of said receptor comprising SEQ ID NO:1 or a functional fragment or functional derivative thereof; and a beta chain of said receptor comprising SEQ ID NO:2 or a functional fragment or functional derivative thereof. In a specific embodiment, the immune cell is a T cell. In some embodiments, the cell comprises an antigen recognition moiety that is not the T cell receptor. In specific embodiments, the antigen recognition moiety is a chimeric antigen receptor, an engager molecule, or another T cell receptor. The antigen recognition moiety recognizes surviving or a tumor antigen other than survivin, in specific embodiments. In some cases, the cell is autologous to an individual, although it may be allogeneic to an individual. In particular embodiments, the functional fragment of SEQ ID NO:1 has a N-terminal truncation of the sequence of SEQ ID NO:1. In some cases, the N-terminal truncation has 1, 2, 3, 4, 5, 6, 7, 8, 9, 10, 11, 12, 13, 14, 15, 16, 17, 18, 19, 20, or more amino acids truncated from the N-terminus of SEQ ID NO:1. In certain cases, the functional fragment of SEQ ID NO:1 has a C-terminal truncation of the sequence of SEQ ID NO:1. In specific embodiments, the C-terminal truncation has 1, 2, 3, 4, 5, 6, 7, 8, 9, 10, 11, 12, 13, 14, 15, 16, 17, 18, 19, 20, or more amino acids truncated from the C-terminus of SEQ ID NO: A specific embodiment provides that the functional derivative of SEQ ID NO:1 is 70%, 75%, 80%, 85%, 90%, 95%, 97%, 98%, or 99% identical to SEQ ID NO:1. In certain embodiments, the functional fragment of SEQ ID NO:2 has a N-terminal truncation of the sequence of SEQ ID NO:2. In specific embodiments, the N-terminal truncation has 1, 2, 3, 4, 5, 6, 7, 8, 9, 10, 11, 12, 13, 14, 15, 16, 17, 18, 19, 20, or more amino acids truncated from the N-terminus of SEQ ID NO:2. In particular embodiments, the functional fragment of SEQ ID NO:2 has a C-terminal truncation of the sequence of SEQ ID NO:2. In specific embodiments, the C-terminal truncation has 1, 2, 3, 4, 5, 6, 7, 8, 9, 10, 11, 12, 13, 14, 15, 16, 17, 18, 19, 20, or more amino acids truncated from the C-terminus of SEQ ID NO:2. In particular embodiments, the functional derivative of SEQ ID NO:2 is 70%, 75%, 80%, 85%, 90%, 95%, 97%, 98%, or 99% identical to SEQ ID NO:2. In particular embodiments, the functional fragment of SEQ ID NO:1 has an N-terminal and a C-terminal truncation of the sequence of SEQ ID NO:1. In some cases, the functional fragment of SEQ ID NO:2 has an N-terminal and a C-terminal truncation of the sequence of SEQ ID NO:2.

In particular embodiments of the composition, the cell expresses a suicide gene product. In specific embodiments, the receptor is HLA-A2 restricted, and the receptor recognizes an epitope selected from the group consisting of an epitope comprising SEQ ID NO:15 or a functional fragment or derivative thereof, an epitope comprising SEQ ID NO:16 or a functional fragment or derivative thereof, or both. In specific embodiments, the functional fragment or derivative of the epitope comprises SEQ ID NO:15 is 70, 75, 77, 80, 85, 88, 90, 91, 91, 95, 97, or 99% identical to SEQ ID NO:15.

In certain embodiments, the functional fragment or derivative of the epitope comprises SEQ ID NO:16 is 70, 75, 77, 80, 85, 88, 90, 91, 91, 95, 97, or 99% identical to SEQ ID NO:16. In specific embodiments, the epitope comprises a N-terminal extension or truncation in relation to SEQ ID NO:15. In specific embodiments, the N-terminal and/or C-terminal extension is 1, 2, 3, 4, 5, 6, 7, 8, 9, or 10 or more amino acids. In certain embodiments, the N-terminal and/or C-terminal truncation is 1, 2, 3, 4, or 5 or more amino acids. In particular embodiments, the epitope comprises a N-terminal extension or truncation in relation to SEQ ID NO:16. In certain embodiments, the N-terminal and/or C-terminal extension is 1, 2, 3, 4, 5, 6, 7, 8, 9, or 10 or more amino acids. In specific cases, the N-terminal and/or C-terminal truncation is 1, 2, 3, 4, or 5 or more amino acids.

In one embodiment, there is a method of providing cell therapy to an individual in need thereof, comprising the step of providing a therapeutically effective amount of any composition contemplated herein to the individual. In specific embodiments, the individual has survivin-positive cancer. In particular embodiments, the survivin-positive cancer is leukemia, myeloma, breast cancer, lung cancer, colon cancer, melanoma, lymphoma, ovarian cancer, prostate cancer, central nervous system cancer, or renal cancer. In specific embodiments, the method may further comprise the step of providing an additional cancer therapy to the individual. In specific embodinents, the additional cancer therapy is chemotherapy, immunotherapy, radiation, surgery, or hormone therapy.

In one embodiment, there is a polynucleotide that expresses the amino acid sequence of SEQ ID NO:1, SEQ ID NO:2, a combination of SEQ ID NO:1 and SEQ ID NO:2, or SEQ ID NO:3. In specific embodiments, the polynucleotide is an expression vector.

In a certain embodiment, there is a cell comprising any polynucleotide as contemplated herein.

In a particular embodiment, there is a kit comprising any composition as contemplated herein, any polynucleotide as contemplated herein, a cell as contemplated herein, or a combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1. Survivin-specific T-cell clone with antitumor effects in the absence of toxicity. (A) FACS analysis of the survivin-specific T-cell clone stained for CD8 and the LML-specific or irrelevant tetramer. (B) T-cell avidity assessed by Interferon-γ (IFN-γ) ELISpot assays of the irrelevant clone against the LML peptide (black bars), and of the survivin-specific clone against the LML (gray bars) or the ELT peptides (white bars). Spot forming cells (SFCs)/105 cells, mean±SD of triplicates. (C) T-cell avidity assessed by 51Cr-release assay against LML-(squares, solid line) or ELT-pulsed T2 cells (triangles, dashed line). Shown are mean±SD of triplicates of the specific lysis at 10:1 E:T ratio. (D) Antitumor activity by 51Cr-release assay of an irrelevant (left panel) and a survivin-specific (right panel) clone derived from the same donor against the HLA-A*02+survivin+ target cell lines BV173 and U266, and the HLA-A*02−survivin+ target cell line HL-60. Shown are mean±SD of triplicates of 1 representative of 2 experiments. (E) Anti-leukemic activity and absence of toxicity against normal hematopoietic progenitors by CFU assay of the survivin-specific clone (open squares) and the irrelevant clone (black circles) against HLA-A*02+survivin+ primary leukemic blasts from two CML blast crisis patients and HLA-A*02+ normal donor bone marrow (BM) donor. Shown is the summary (mean±SD) of 3 independent experiments in duplicates, * p<0.001, ** p=0.001. (F) Absence of T-cell fratricide, assessed as fold expansion over 3 weeks of culture after superexpansion of the survivin-specific (open squares, dashed line) and irrelevant (black circles, solid line) clones.

FIG. 2. Efficient expression of the transgenic survivin TCR by polyclonal CD8+ T cells. (A) Scheme of the retroviral vector. (B) Transduction efficiency detected by staining for the murine constant β chain (mCβ) and LML-tetramer. Enrichment of LML-tetramer+ cells during T-cell expansion in the presence of LML-pulsed aAPCs (2 weekly stimulations). Representative FACS plots (left) immediately after transduction (after TD), and after one (End 51) or two stimulations (End S2). Graph on the right shows the mean±SD of 4 donors. (C) Increase in the LML-tetramer mean fluorescence intensity (MFI) after weekly antigen-specific stimulations. Representative histogram (left) staining with irrelevant tetramer (gray), LML tetramer after TD (black), End 51 (blue) and End S2 (red). The graph (right) shows the mean±SD of 4 donors.

FIG. 3. The ectopically expressed survivin TCR is functional and specific, but not fratricidal in vitro. (A-C, E, F) Symbols represent means of triplicates/donor, horizontal bars means±SD. (A) Production of IFN-γ (ELISpot) in response to LML and ELT peptides by non-transduced (NT, black circles) and transduced (TD, open squares) T cells, n=5. (B) Killing of LML-pulsed T2 cells (51Cr-release) by NT and TD T cells, % specific lysis at E:T 20:1, n=3. (C) Evaluation of HLA restriction of the killing of LML-pulsed T2 cells by preincubation with HLA class I (dotted line), HLA class II blocking antibody (dashed line) or in the absence of antibody (solid line) by TD T cells (white squares) and NT T cells (black squares, solid line). 1 representative of 2 experiments. (D) Expansion of transgenic T cells with weekly antigen-specific stimulations generated from HLA-A*02+ (black circles, solid line) or HLA-A*02− (open squares, dashed line) donors. Mean±SD, n=7, p=NS. (E and F). 51Cr-release assays of NT (black circles) and TD T cells (white squares) against activated HLA-A*02+ target T cells loaded or not with LML or ELT peptide. % specific lysis at E:T 20:1 of survivin TCR expressed in (E) HLA-A*02− (n=7) or (F) HLA-A*02+ donors (n=7).

FIG. 4. Survivin-TCR redirected T cells have antitumor activity in vitro while lack toxicity against normal hematopoietic stem/progenitor cells. (A) 51Cr-release assays by survivin TCR+TD (open squares) and NT control T cells (black circles) against HLA-A*02+survivin+ cancer cell lines BV173 and U266, and the HLA-A*02−survivin+ targets HL-60 and K562. Symbols: means of triplicates/donor, bars: mean±SD of specific lysis (E:T 20:1), n=12 donors. *p<0.001, **p=0.003. (B) HLA restriction of TCR+ TD (open symbols) and NT T cells (black symbols) assessed by preincubation of BV173 (left panel, triangles) or U266 (right panel, circles) with HLA class I blocking antibody (dotted lines), HLA class II blocking antibody (dashed lines) or in the absence of antibody (no Ab, solid lines). Mean±SD of triplicates, 1 representative of 2 donors. (C) Quantification of residual tumor cells in co-cultures on day 5 of TCR+ TD (open squares) and NT (black circles) T cells cultured with BV173, U266, K562 and HL-60 cells (E:T 5:1). Mean±SD of residual tumor cells, n=6. * p<0.001, ** p=0.02. (D) IFN-γproduction by TCR+ TD (open squares) and NT (black circles) T cells against BV173, U266, K562 and HL-60 cells by ELISpot. Symbols: means of triplicates/donor, bars: mean±SD, n=5. *p<0.001, **p=0.01. (E, F, G) Assessment of leukemic colony formation by TCR+ TD (open squares) and NT (black circles) T cells against HLA-A*02+ CML blast crisis (n=2) and AML (n=3) (E), HLA-A*02− leukemic blasts (F), and HLA-A*02+ healthy donor-derived bone marrow (BM, n=1) or cord blood (CB, n=4) progenitors (G). Mean±SD of CFUs for 5 donors plated in duplicates. *p<0.001.

FIG. 5. Survivin-TCR redirected T cells have in vivo antileukemic activity. (A) Experimental plan. Intravenous administration of 3×10⁶ BV173-FFluc cells to NSG mice after sublethal irradiation (120cGy), followed by T cell infusions, IL2 and weekly bioluminescent imaging (BLI) starting on day 18. (B) Time-course of BLI in representative individual mice from both treatment groups, scale 5×10⁴ to 5×10⁵ photons/sec/cm²/sr. (C) Average photons/sec/cm²/sr per mouse, determined by BLI, comparing mice treated with control T cells (NT, n=9, black circles) or survivin TCR⁺ T cells (TD, n=10, open squares). Mean±SD, *p=0.01 at day 32 and 0.009 at day 39, after adjustment for multiple comparisons. The intensity signals were also log-transformed and the response profiles over time were analyzed by the robust generalized estimating equations method (p<0.0001). Summary of 2 independent experiments. (D) Kaplan-Meier survival curve of mice treated with survivin TCR⁺ T cells (TD) or control T cells (NT) (p<0.001).

FIG. 6. Survivin-TCR+ T cells prolong survival of mice with high leukemia burden. (A) Experimental plan. Intravenous administration of 3×10⁶ BV173-FFluc cells to NSG mice after sublethal irradiation (120cGy). T cells were infused 14 to 17 days later, when leukemia was disseminated and established in multiple organs as detected by BLI. T-cell infusions, IL-2 and weekly BLI. (B) Time-course of BLI in representative individual mice from both treatment groups, scale 1×10³ to 1×10⁴ photons/sec/cm²/sr (day 0), 1×10⁵ to 1×10⁶ photons/sec/cm²/sr (days 7-28). (C) Average photons/sec/cm²/sr per mouse comparing mice treated with control T cells (NT, n=16) or survivin-TCR⁺ T cells (TD, n=15). Mean±SD. The intensity signals were also log-transformed and the response profiles over time were analyzed by the robust generalized estimating equations method (p=0.006). Summary of 3 independent experiments. (D) Kaplan-Meier survival curve of mice treated with survivin TCR⁺ T cells (TD) or control T cells (NT) (p=0.01).

FIG. 7. Fratricidal activity of allogeneic repertoire derived survivin TCR. (A-E) Comparison of TCR+ T cells transduced with s24-survivin TCR (s24-TD, white bars) with the A72 survivin-TCR (A72-TD, gray bars) or NT control T cells (black bars). 1 representative of 2-4 donors, mean±SD of triplicates. (A) 51Cr-release assay against HLA-A2+survivin+ (BV173, U266) and HLA-A2-survivin+ (HL-60, K562) cancer cell lines. Mean±SD of triplicates for specific lysis (E:T 20:1). (B) 51Cr-release assay against activated HLA-A*0201+ target T cells in the absence of exogenous peptide (−) or pulsed with LML or ELT peptide. Mean±SD % specific lysis of triplicates, E:T 20:1. (C) CFU assay with normal HLA-A2+ cord blood donors. Mean of duplicates, E:T 10:1. 51Cr-release assay against HLA-A*0201+ fibroblasts (D) and the HLA-A*0201+ cardiomyocyte cell line AC10 (E) with IFN-γ pretreatment or IFN-γ pretreatment and pulsed with LML peptide. Mean±SD % specific lysis, summary of 4 donors, E:T 20:1.

FIG. 8. Different molecular recognition patterns of autologous versus allogeneic repertoire derived survivin-TCRs. Alanine-substitution analysis testing s24 TD (white bars) or A72 TD (gray bars) T cells for recognition of peptide-pulsed T2 cells by IFN-γ ELISpot. Mean±SD, n=4 donors.

FIG. 9. Transgenic TCR expression in HLA-A2+ and HLA-A2− donors is comparable. Survivin-TCR transduced CD8+ T cells from HLA-A*02+ (black circles) and HLA-A*02− (open squares) healthy adult donors after 2 antigen-specific stimulations were compared for transduction efficiency and tetramer mean fluorescence intensity (MFI). (A) Percentage of mCβ+ and LML-tetramer+ cells and (B) MFI of LML-tetramer in HLA-A2+ and HLA-A2− transduced T cells. Mean±SD, n=5.

FIG. 10. Representative FACS analysis of co-cultures. Coculture of control T cells (NT, top row) or survivin TCR+ T cells (TD, lower row) with HLA-A*02+survivin+ (BV173, U266) or HLA-A*02−survivin+ (HL-60, K562) cancer cell lines at an E:T ratio of 5:1 in the absence of cytokines. FACS analysis on day 5 shows staining for CD3 (T cells) and the tumor markers CD19 (BV173), CD138 (U266), CD33 (HL-60 and K562). Shown is 1 experiment representative of 8 donors.

FIG. 11. Cytokine production of TCR+ T cells in co-culture. Analysis by cytometric bead array (CBA) of supernatant collected after 24 hours from co-cultures to determine the concentrations (pg/ml) of Interferon-γ (IFN-γ), Tumor Necrosis Factor-α (TNF-α), IL10, IL4 and IL2 by TCR+ T cells (TD, white bars) and control (NT, black bars). Shown is 1 experiment representative of 2 donors.

FIG. 12. TCRs derived from autologous repertoires have lower potential for cross-reactivity. Alanine-substitution analysis of T cells engineered to express transgenic autologous TCRs (A) and allogeneic TCRs (B) for recognition of peptide-pulsed T2 cells by IFN-γ ELISpot when targeting different TAAs. Mean±SD of triplicates, 1 representative of 2 donors tested for each TCR.

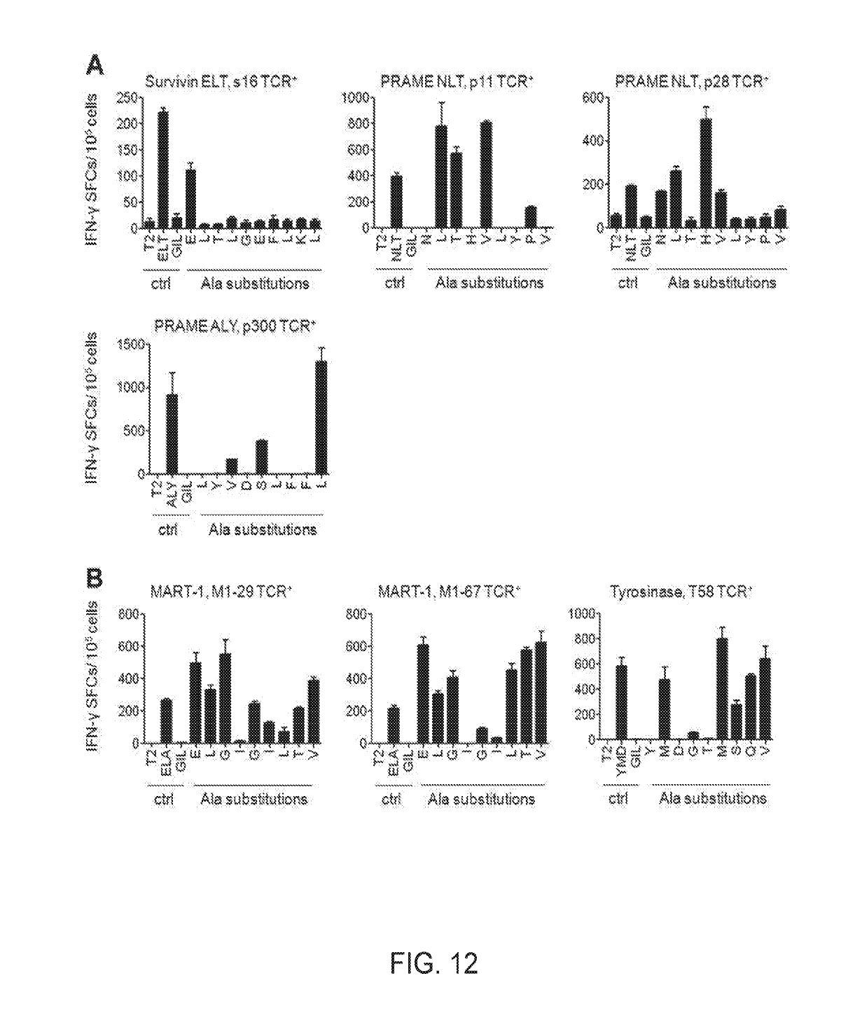

FIG. 13. HLA-A2 and survivin expression of fibroblasts and cardiomyocytes. FACS analysis of fibroblasts (A) and the cardiomyocyte cell line AC10 (B) for HLA-A2 (surface) and survivin (intracellular) without (gray line) or with (black line) IFN-γ treatment. Isotype control (black line, shaded area).

FIG. 14. Anti-tumor activity of s24− versus A72−TCR+ T cells in vivo in the BV173 mouse model. Same experimental plan as depicted in FIG. 5(A) comparing anti-tumor activity of s24-TCR+ T cells (n=15) and A72-TCR+ T cells (n=10) in mice by BLI. The intensity signals were log-transformed and the response profiles over time were analyzed using the robust generalized estimating equations method (p<0.0001).

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein embodiments which are disclosed and still obtain a like or similar result without departing from the spirit and scope of the invention.

Survivin is a broadly expressed tumor-associated antigen exerting crucial functions in cancer cells. Immunotherapeutic targeting of this antigen using transgenic T-cell receptors (TCRs) has however been impeded by the “fratricide” activity observed in T cells expressing high-avidity survivin-specific TCRs isolated from allogeneic HLA-mismatched TCR repertoires. Herein it is shown that an HLA-A2-restricted survivin-specific TCR with antitumor activity in vitro and in vivo but lacking fratricidal toxicity can be isolated when starting from autologous TCR repertoires. To understand the mechanistic basis of this selective activity alanine-scanning was performed, revealing that the autologous-derived TCR had a more specific interaction with the surviving peptide as compared to a “fratricide” TCR. Thus, maximal peptide recognition is key for TCR selectivity and may be critical in reducing unwanted off-target toxicities. This strategy may be adapted to identify and select other shared tumor/self-antigen-specific TCRs that will possess selective antitumor activity.

### I. Exemplary Survivin-Specific T Cell Receptors

The present disclosure concerns T cell receptors (TCR) having particular alpha and beta chains, wherein the T cell is specific for the survivin antigen. In particular aspects, the receptor is an engineered receptor by the hand of man. The receptor is recombinantly produced, in particular embodiments, and is expressed on an immune cell, such as a T cell. The receptor is capable of recognizing the survivin antigen on cancer cells and, in specific embodiments, the receptor does not recognize the survivin antigen on non-cancer cells or recognizes it at a reduced level compared to cancer cells.

In specific embodiments, the TCR comprises an alpha chain that comprises SEQ ID NO:1 or a functional fragment or functional derivative thereof. In specific embodiments, the TCR comprises a beta chain that comprises SEQ ID NO:2 or a functional fragment or functional derivative thereof. In specific embodiments, the TCR comprises both an alpha chain that comprises SEQ ID NO:1 or a functional fragment or functional derivative thereof and a beta chain that comprises SEQ ID NO:2 or a functional fragment or functional derivative thereof.

In embodiments of the disclosure, the T cell receptors bind to one or more epitopes on survivin. Although the epitope may be of any kind, in specific embodiments the epitope comprises, consists of, or consists essentially of SEQ ID NO:15 or SEQ ID NO:16 or a fragment thereof, including a functional fragment thereof. In particular embodiments, the epitope that is recognized by the T cell receptor of the disclosure is or is at least 70, 75, 77, 80, 85, 88, 90, 91, 91, 95, 97, or 99% identical to SEQ ID NO:15 or SEQ ID NO:16 or a fragment thereof, including a functional fragment thereof. The epitope may have N-terminal and/or C-terminal extensions or truncation in relation to SEQ ID NO:15 or SEQ ID NO:16. Such N-terminal and/or C-terminal extensions may be 1, 2, 3, 4, 5, 6, 7, 8, 9, or 10 or more amino acids. Such N-terminal and/or C-terminal truncations may be 1, 2, 3, 4, or 5 or more amino acids. The TCRs of the disclosure may be able to bind survivin epitopes that have 1, 2, 3, 4, 5, or more alterations at particular residues compared to SEQ ID NO:15 or SEQ ID NO:16. In specific embodiments, Leu4, Gly5 and/or Phe7 of SEQ ID NO:15 are not altered, although in alternative embodiments one or more of them are altered.

A. Proteinaceous Compositions, Generally

In certain embodiments, the present invention concerns novel TCR compositions comprising at least one proteinaceous molecule. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino molecule” refers to any amino acid, amino acid derivitive or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's GenBank® and GenPept® databases (http://www.ncbi.nlm.nih.gov/). The coding regions for these known genes may be amplified and/or expressed using the techniques disclosed herein or as would be known to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism, with a selected animal or human subject being preferred. The “autologous protein, polypeptide or peptide” may then be used as a component of a composition intended for application to the selected animal or human subject. In certain aspects, the autologous proteins or peptides are prepared, for example from whole plasma of the selected donor. The plasma is placed in tubes and placed in a freezer at about −80° C. for at least about 12 hours and then centrifuged at about 12,000 times g for about 15 minutes to obtain the precipitate. The precipitate, such as fibrinogen may be stored for up to about one year (Oz, 1990).

B. Biological Functional Equivalents

As modifications and/or changes may be made in the structure of the TCR polynucleotides and and/or proteins according to the present invention, while obtaining molecules having similar or improved characteristics, such biologically functional equivalents are also encompassed within the present invention.

The disclosure provides TCR alpha and beta chains that are fragments and/or derivatives of SEQ ID NO:1 and SEQ ID NO:2, respectively. Such fragments and/or derivatives will maintain the activity of SEQ ID NO:1 and SEQ ID NO:2, respectively. In particular embodiments, the fragments and/or derivatives as part of a TCR in its totality will provide selective antitumor activity.

1. Modified Polynucleotides and Polypeptides

The biological functional equivalent may comprise a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, a polynucleotide may be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted. In specific embodiments, functional activity includes selective antitumor activity, including activity that lacks fratricidal effects or toxicity against normal hematopoietic stem/progenitor cells. The functional activity lacks autotoxicity, in particular embodiments. The functional activity allows discrimination of survivin on self tissues from tumor-associated survivin expression and selectively mediates antitumor reactivity without “on target off-tumor” activity, in certain aspects.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (0.4); threonine (0.7); serine (0.8); tryptophan (0.9); tyrosine (1.3); proline (1.6); histidine (3.2); glutamate (3.5); glutamine (3.5); aspartate (3.5); asparagine (3.5); lysine (3.9); and/or arginine (4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (0.4); proline (−0.5±1); alanine (0.5); histidine (0.5); cysteine (1.0); methionine (1.3); valine (1.5); leucine (1.8); isoleucine (1.8); tyrosine (2.3); phenylalanine (2.5); tryptophan (3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

2. Altered Amino Acids

The present invention, in many aspects, relies on the synthesis of peptides and polypeptides in cyto, via transcription and translation of appropriate polynucleotides. These peptides and polypeptides will include the twenty “natural” amino acids, and post-translational modifications thereof. However, in vitro peptide synthesis permits the use of modified and/or unusual amino acids. Exemplary, but not limiting, modified and/or unusual amino acids is as follows: 2-Aminoadipic acid, N-Ethylasparagine, 3-Aminoadipic acid, Hydroxylysine, beta-alanine, beta-Amino-propionic acid, allo-Hydroxylysine, 2-Aminobutyric acid, 3-Hydroxyproline, 4-Aminobutyric acid, piperidinic acid, 4-Hydroxyproline, 6-Aminocaproic acid, Isodesmosine, 2-Aminoheptanoic acid, allo-Isoleucine, 2-Aminoisobutyric acid, N-Methylglycine , sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

3. Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins. Vita et al. (1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids. Weisshoff et al. (1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. These structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

4. Specific Embodiments

An example of a survivin-specific T cell receptor comprising a beta chain, an alpha chain, multiple framework regions, multiple diversity regions, a joining region, and constant regions of TCRalpha and TCRbeta of mouse origin is provided in SEQ ID NO:3. An example of a polynucleotide that encodes a a survivin-specific T cell receptor comprising a beta chain, an alpha chain, multiple framework regions, multiple diversity regions, a joining region, and constant regions of TCRalpha and TCRbeta of mouse origin is provided in SEQ ID NO:5. An example of a vector that encodes a beta chain, an alpha chain, multiple framework regions, multiple diversity regions, multiple joining regions, and constant regions of TCRalpha and TCRbeta of mouse origin is provided in SEQ ID NO:4; said polynucleotide also comprises 5′LTR and 3′LTR.

### II. Host Cells Expressing Survivin-Specific TCRs

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T-cell (also known as TC, Cytotoxic T Lymphocyte, CTL, T-Killer cell, cytolytic T cell, CD8+ T-cells, CD4+ T-cells, or killer T-cells); NK cells and NKT cells are also encompassed in the invention.

In one aspect, provided herein is a cell that has been genetically engineered to express one or more TCRs. In certain embodiments, the genetically engineered cell is, e.g., a T lymphocyte (T-cell), a natural killer (NK) T-cell, or an NK cell. In certain other embodiments, the genetically engineered cell is a non-immune cell, e.g., a mesenchymal stem cell (MSC), a neuronal stem cell, a hematopoietic stem cell, an induced pluripotent stem cell (iPS cell), or an embryonic stem cell, for example. In specific embodiments, the cell also comprises an engineered TCR or any other genetic modification that may enhance its function.

In certain embodiments, it is contemplated that RNAs or proteinaceous sequences may be co expressed with other selected RNAs or proteinaceous sequences in the same cell, such as the same CTL. Co expression may be achieved by co transfecting the CTL with two or more distinct recombinant vectors. Alternatively, a single recombinant vector may be constructed to include multiple distinct coding regions for RNAs, which could then be expressed in CTLs transfected with the single vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the genetically engineered T-cells, where one wishes to terminate the treatment, the cells become neoplastic, in research where the absence of the cells after their presence is of interest, or other purpose. For this purpose one can provide for the expression of certain gene products in which one can kill the engineered cells under controlled conditions, such as inducible suicide genes. Such suicide genes are known in the art, e.g., the iCaspase9 system in which a modified form of caspase 9 is dimerizable with a small molecule, e.g., AP1903. See, e.g., Straathof et al., Blood 105:4247-4254 (2005).

It is further envisaged that the pharmaceutical composition of the disclosure comprises a host cell transformed or transfected with a vector defined herein. The host cell may be produced by introducing at least one of the above described vectors or at least one of the above described nucleic acid molecules into the host cell. The presence of the at least one vector or at least one nucleic acid molecule in the host may mediate the expression of a gene encoding the above described be specific single chain antibody constructs.

The described nucleic acid molecule or vector that is introduced in the host cell may either integrate into the genome of the host or it may be maintained extrachromosomally.

The host cell can be any prokaryote or eukaryotic cell, but in specific embodiments it is a eukaryotic cell. In specific embodiments, the host cell is a bacterium, an insect, fungal, plant or animal cell. It is particularly envisaged that the recited host may be a mammalian cell, more preferably a human cell or human cell line. Particularly preferred host cells comprise immune cells, CHO cells, COS cells, myeloma cell lines like SP2/0 or NS/0.

The pharmaceutical composition of the disclosure may also comprise a proteinaceous compound capable of providing an activation signal for immune effector cells useful for cell proliferation or cell stimulation. In the light of the present disclosure, the “proteinaceous compounds” providing an activation signal for immune effector cells may be, e.g. a further activation signal for T-cells (e.g. a further costimulatory molecule: molecules of the B7-family, OX40 L, 4-1BBL), or a further cytokine: interleukin (e.g. IL-2, IL-7, or IL-15), or an NKG-2D engaging compound. The proteinaceous compound may also provide an activation signal for immune effector cell which is a non-T-cell. Examples for immune effector cells which are non-T-cells comprise, inter alia, NK cells, or NKT-cells.

One embodiment relates to a process for the production of a composition of the disclosure, the process comprising culturing a host cell defined herein above under conditions allowing the expression of the construct, and the cell or a plurality of cells is provided to the individual.

The conditions for the culturing of cells harboring an expression construct that allows the expression of the TCR molecules are known in the art, as are procedures for the purification/recovery of the constructs when desired.

In one embodiment, the host cell is a genetically engineered T-cell (e.g., cytotoxic T lymphocyte) comprising a TCR that has a high-avidity and reactivity toward target antigens that is selected, cloned, and/or subsequently introduced into a population of T-cells used for adoptive immunotherapy.

### III. Pharmaceutical Compositions

Provided herein are pharmaceutical compositions comprising the genetically engineered immune cells, e.g., genetically engineered survivin-specific TCR-expressing T cells.

In accordance with this disclosure, the term “pharmaceutical composition” relates to a composition for administration to an individual. In a preferred embodiment, the pharmaceutical composition comprises a composition for parenteral, transdermal, intraluminal, intra-arterial, intrathecal or intravenous administration or for direct injection into a cancer. It is in particular envisaged that said pharmaceutical composition is administered to the individual via infusion or injection. Administration of the suitable compositions may be effected by different ways, e.g., by intravenous, subcutaneous, intraperitoneal, intramuscular, topical or intradermal administration.

The pharmaceutical composition of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. An example of a dosage for administration might be in the range of An example of a dosage for administration might range from 2×10⁷ cells/m² of body surface area or 1×10⁶ cells/Kg body weight up to 2×10⁸ cells/m² or 5×10⁶ cells/Kg. These infusions may be repeated. Progress can be monitored by periodic assessment.

The TCR cell compositions of the disclosure may be administered locally or systemically. Administration will generally be parenteral, e.g., intravenous; DNA may also be administered directly to the target site, e.g., by biolistic delivery to an internal or external target site or by catheter to a site in an artery. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the pharmaceutical composition.

### V. Therapeutic Uses of TCRs and Host T-cells Comprising TCRs

In various embodiments, TCR constructs, nucleic acid sequences, vectors, host cells , as contemplated herein and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancer having tumors, for example.

In particular embodiments, provided herein is a method of treating an individual for cancer, comprising the step of providing a therapeutically effective amount of a plurality of any of cells of the disclosure to the individual. In certain aspects, the cancer is a solid tumor, and the tumor may be of any size. In certain aspects, the method further comprises the step of providing a therapeutically effective amount of an additional cancer therapy to the individual.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present invention contemplates, in part, cells, TCR constructs, nucleic acid molecules and vectors that can administered either alone or in any combination using standard vectors and/or gene delivery systems, and in at least some aspects, together with a pharmaceutically acceptable carrier or excipient. In certain embodiments, subsequent to administration, said nucleic acid molecules or vectors may be stably integrated into the genome of the subject.

In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject or individual in the need thereof an effective amount of immune cells, e.g., T cells or cytotoxic T lymphocytes, harboring a survivin-specific TCR-expressing cell; a nucleic acid sequence encoding the TCR; a vector comprising a nucleotide sequence encoding the TCR, as described herein and/or produced by a process as described herein.

Possible indications for administration of the composition(s) of the exemplary TCR cells are cancerous diseases, including tumorous diseases, including breast, prostate, lung, and colon cancers or epithelial cancers/carcinomas such as breast cancer, colon cancer, prostate cancer, head and neck cancer, skin cancer, cancers of the genitourinary tract, e.g. ovarian cancer, endometrial cancer, cervical cancer and kidney cancer, lung cancer, gastric cancer, cancer of the small intestine, liver cancer, pancreatic cancer, gall bladder cancer, cancers of the bile duct, esophagus cancer, cancer of the salivary glands and cancer of the thyroid gland. The administration of the composition(s) of the disclosure is useful for all stages and types of cancer, including for minimal residual disease, early cancer, advanced cancer, and/or metastatic cancer and/or refractory cancer, for example, wherein the cancer is associated with pathogenic vascularization.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Particular doses for therapy may be determined using routine methods in the art. However, in specific embodiments, the T cells are delivered to an individual in need thereof once, although in some cases it is multiple times, including 2, 3, 4, 5, 6, or more times. When multiple doses are given, the span of time between doses may be of any suitable time, but in specific embodiments, it is weeks or months between the doses. The time between doses may vary in a single regimen. In particular embodiments, the time between doses is 2, 3, 4, 5, 6, 7, 8, 9, 10, or more weeks. In specific cases, it is between 4-8 or 6-8 weeks, for example.

In particular embodiments, there are pharmaceutical compositions that comprise cells that express survivin-specific TCR. An effective amount of the cells are given to an individual in need thereof.

By way of illustration, cancer patients or patients susceptible to cancer or suspected of having cancer may be treated as follows. Cells modified as described herein may be administered to the patient and retained for extended periods of time. The individual may receive one or more administrations of the cells. In some embodiments, the genetically engineered cells are encapsulated to inhibit immune recognition and placed at the site of the tumor.

In particular cases, the individual is provided with therapeutic T-cells engineered to comprise a TCR specific for survivin. In multiple iterations of the delivery, the cells may be delivered in the same or separate formulations. The cells may be provided to the individual in separate delivery routes. The cells may be delivered by injection at a tumor site or intravenously or orally, for example. Routine delivery routes for such compositions are known in the art.

Expression vectors that encode the TCRs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the TCR and optionally another gene, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

The exemplary T cells that have been engineered to include the TCR construct(s) are then grown in culture under selective conditions and cells that are selected as having the construct may then be expanded and further analyzed, using, for example; the polymerase chain reaction for determining the presence of the construct in the host cells. Once the engineered host cells have been identified, they may then be used as planned, e.g. expanded in culture or introduced into a host organism.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that the system is subject to many variables, such as the cellular response to the ligand, the efficiency of expression and, as appropriate, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

In another aspect, provided herein is a method of treating an individual having a tumor cell, comprising administering to the individual a therapeutically effective amount of cells expressing at least TCR. In a related aspect, provided herein is a method of treating an individual having a tumor cell, comprising administering to the individual a therapeutically effective amount of cells expressing at least the survivin-specific TCR. In a specific embodiment, said administering results in a measurable decrease in the growth of the tumor in the individual. In another specific embodiment, said administering results in a measurable decrease in the size of the tumor in the individual. In various embodiments, the size or growth rate of a tumor may be determinable by, e.g., direct imaging (e.g., CT scan, MRI, PET scan or the like), fluorescent imaging, tissue biopsy, and/or evaluation of relevant physiological markers (e.g., PSA levels for prostate cancer; HCG levels for choriocarcinoma, and the like). In specific embodiments of the invention, the individual has a high level of an antigen that is correlated to poor prognosis. In some embodiments, the individual is provided with an additional cancer therapy, such as surgery, radiation, chemotherapy, hormone therapy, immunotherapy, or a combination thereof.

IV. Kits of the Disclosure

Embodiments relate to a kit comprising cells as defined herein, TCR constructs as defined herein, a nucleic acid sequence as defined herein, and/or a vector as defined herein. It is also contemplated that the kit of this disclosure comprises a pharmaceutical composition as described herein above, either alone or in combination with further medicaments to be administered to an individual in need of medical treatment or intervention.

Any of the compositions described herein may be comprised in a kit. In a non-limiting example, cells for cell therapy or one or more reagents to produce the cells may be comprised in a kit. The kits will thus comprise, in suitable container means, cells, vectors, primers, enzymes, buffers, salts, nucleotides, polynucleotides, and so forth may be comprised in a kit. In particular embodiments, the cells have been transduced with a particular vector encoding a TCR specific for survivin, including a TCR comprising one or both of an alpha chain of said receptor comprising SEQ ID NO:1 or a functional fragment or functional derivative thereof; and a beta chain of said receptor comprising SEQ ID NO:2 or a functional fragment or functional derivative thereof. The kits may also comprise bacteria for further manipulation of the receptor or a polynucleotide encoding same. The kits may comprise untransduced mammalian immune cells, such as immune cells capable of being transduced with a vector encoding part or all of a TCR of the disclosure. The kits may comprise polynucleotides that encode part or all of a TCR of the disclosure, including a vector polynucleotide, such as one that comprise an expression construct.

The kits may comprise a suitably aliquoted cell compositions of the present disclosure, or suitably aliquoted reagents to generate cells. The components of the kits may be packaged either in aqueous media or in lyophilized form. The container means of the kits may generally include at least one vial, test tube, flask, bottle, syringe or other container means, into which a component may be placed, and preferably, suitably aliquoted. The kits may also comprise a second container means for containing a sterile, pharmaceutically acceptable buffer and/or other diluent. Where there are more than one component in the kit, the kit also will generally contain a second, third or other additional container into which the additional component(s) may be separately placed. However, various combinations of components may be comprised in a vial. The kit may have a single container means, and/or it may have distinct container means for each compound. The kits of the present invention also will typically include a means for containing any container(s) in close confinement for commercial sale. Such containers may include injection or blow-molded plastic containers into which the desired vials are retained.

Kits of the present disclosure may comprise polynucleotides that encode part or all of a T cell receptor and/or primers to produce such polynucleotides, such as by amplification. Such polynucleotides may encode the T cell receptor beta chain, T cell receptor alpha chain, or another region of the receptor, for example.

When the components of the kit are provided in one and/or more liquid solutions, the liquid solution may be an aqueous solution, with a sterile aqueous solution being particularly preferred. The compositions may also be formulated into a deliverable composition. In which case, the container means may itself be a syringe, pipette, and/or other such like apparatus, from which the formulation may be applied to an infected area of the body, injected into an animal, and/or even applied to and/or mixed with the other components of the kit.

However, in certain embodiments the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the ultimate Such an instrument may be a syringe, pipette, forceps, and/or any such medically approved delivery vehicle.

In certain embodiments of the disclosure, the kit includes one or more apparatuses or reagents for diagnosis of a particular type of cancer. In particular embodiments of the disclosure, the kit includes one or more additional therapies for cancer, such as chemotherapy, for example.

### V. Polynucleotides Encoding TCRs

The present disclosure also encompasses a composition comprising a nucleic acid sequence encoding a TCR as defined herein and cells harboring the nucleic acid sequence. The nucleic acid molecule is a recombinant nucleic acid molecule, in particular aspects and may be synthetic. It may comprise DNA, RNA as well as PNA (peptide nucleic acid) and it may be a hybrid thereof.

It is evident to the person skilled in the art that one or more regulatory sequences may be added to the nucleic acid molecule comprised in the composition of the disclosure. For example, promoters, transcriptional enhancers and/or sequences that allow for induced expression of the polynucleotide of the disclosure may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62), or a dexamethasone-inducible gene expression system as described, e.g. by Crook (1989) EMBO J. 8, 513-519.

Furthermore, it is envisaged for further purposes that nucleic acid molecules may contain, for example, thioester bonds and/or nucleotide analogues. The modifications may be useful for the stabilization of the nucleic acid molecule against endo- and/or exonucleases in the cell. The nucleic acid molecules may be transcribed by an appropriate vector comprising a chimeric gene that allows for the transcription of said nucleic acid molecule in the cell. In this respect, it is also to be understood that such polynucleotides can be used for “gene targeting” or “gene therapeutic” approaches. In another embodiment the nucleic acid molecules are labeled. Methods for the detection of nucleic acids are well known in the art, e.g., Southern and Northern blotting, PCR or primer extension. This embodiment may be useful for screening methods for verifying successful introduction of the nucleic acid molecules described above during gene therapy approaches.

The nucleic acid molecule(s) may be a recombinantly produced chimeric nucleic acid molecule comprising any of the aforementioned nucleic acid molecules either alone or in combination. In specific aspects, the nucleic acid molecule is part of a vector.

The present disclosure therefore also relates to a composition comprising a vector comprising the nucleic acid molecule described in the present disclosure.

Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994). Alternatively, the polynucleotides and vectors of the disclosure can be reconstituted into liposomes for delivery to target cells. A cloning vector may be used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

In specific embodiments, there is a vector that comprises a nucleic acid sequence that is a regulatory sequence operably linked to the nucleic acid sequence encoding a TCR construct defined herein. Such regulatory sequences (control elements) are known to the artisan and may include a promoter, a splice cassette, translation initiation codon, translation and insertion site for introducing an insert into the vector. In specific embodiments, the nucleic acid molecule is operatively linked to said expression control sequences allowing expression in eukaryotic or prokaryotic cells.

It is envisaged that a vector is an expression vector comprising the nucleic acid molecule encoding a TCR construct defined herein. In specific aspects, the vector is a viral vector, such as a lentiviral vector. Lentiviral vectors are commercially available, including from Clontech (Mountain View, Calif.) or GeneCopoeia (Rockville, Md.), for example.

The term “regulatory sequence” refers to DNA sequences that are necessary to effect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the recited vector is an expression vector, in certain embodiments. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements that are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al. Cancer Immunol Immunother (2001) 50(3), 141-150) or pSPORT1 (GIBCO BRL).

In some embodiments, the expression control sequences are eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the disclosure may follow.

Additional regulatory elements may include transcriptional as well as translational enhancers. Advantageously, the above-described vectors of the disclosure comprises a selectable and/or scorable marker. Selectable marker genes useful for the selection of transformed cells are well known to those skilled in the art and comprise, for example, antimetabolite resistance as the basis of selection for dhfr, which confers resistance to methotrexate (Reiss, Plant Physiol. (Life-Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus that confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or beta-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule can be used in a cell, alone, or as part of a vector to express the encoded polypeptide in cells. The nucleic acid molecules or vectors containing the DNA sequence(s) encoding any one of the TCR constructs described herein is introduced into the cells that in turn produce the polypeptide of interest. The recited nucleic acid molecules and vectors may be designed for direct introduction or for introduction via liposomes, or viral vectors (e.g., adenoviral, retroviral) into a cell. In certain embodiments, the cells are T-cells, TCR T-cells, NK cells, NKT-cells, MSCs, neuronal stem cells, or hematopoietic stem cells, for example.

In accordance with the above, the present disclosure relates to methods to derive vectors, particularly plasmids, cosmids, viruses and bacteriophages used conventionally in genetic engineering that comprise a nucleic acid molecule encoding the polypeptide sequence of a TCR defined herein. In certain cases, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the recited polynucleotides or vector into targeted cell populations. Methods that are well known to those skilled in the art can be used to construct recombinant vectors; see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the disclosure can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

### VI. Combination Therapy

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. In the context of the present invention, it is contemplated that cell therapy could be used similarly in conjunction with chemotherapeutic, radiotherapeutic, or immunotherapeutic intervention, in addition to other pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, present disclosure is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Combination anti-cancer agents include, for example, acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estrarnustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone: didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin: neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer, or any analog or derivative variant of the foregoing and also combinations thereof.

In specific embodiments, chemotherapy for the individual is employed in conjunction with the invention, for example before, during and/or after administration of the invention.

B. Radiotherapy

Other factors that cause DNA damage and have been used extensively include what are commonly known as gamma-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In certain embodiments, the immunotherapy is an antibody against a Notch pathway ligand or receptor, e.g., an antibody against DLL4, Notchl, Notch2/3, Fzd7, or Wnt. In certain other embodiments, the immunotherapy is an antibody against r-spondin (RSPO) 1, RSPO2, RSPO3 or RSPO4.

D. Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR4 or DRS/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Generation of Autologous Survivin-Specific T-Cell Clones With Selective Antitumor Effects

Peripheral blood samples collected from HLA-A*02⁺ healthy donors were used to generate CD8⁺ cytotoxic T cells (CTLs) specific for the HLA-A*0201-restricted survivin₉₅₋₁₀₄ (referred to as ELT; ELTLGEFLKL; SEQ ID NO:15) epitope, using its heteroclitic variant survivin₉₆₋₁₀₄97M (referred to as LML; LMLGEFLKL; SEQ ID NO:16) (Andersen, et al., 2001).

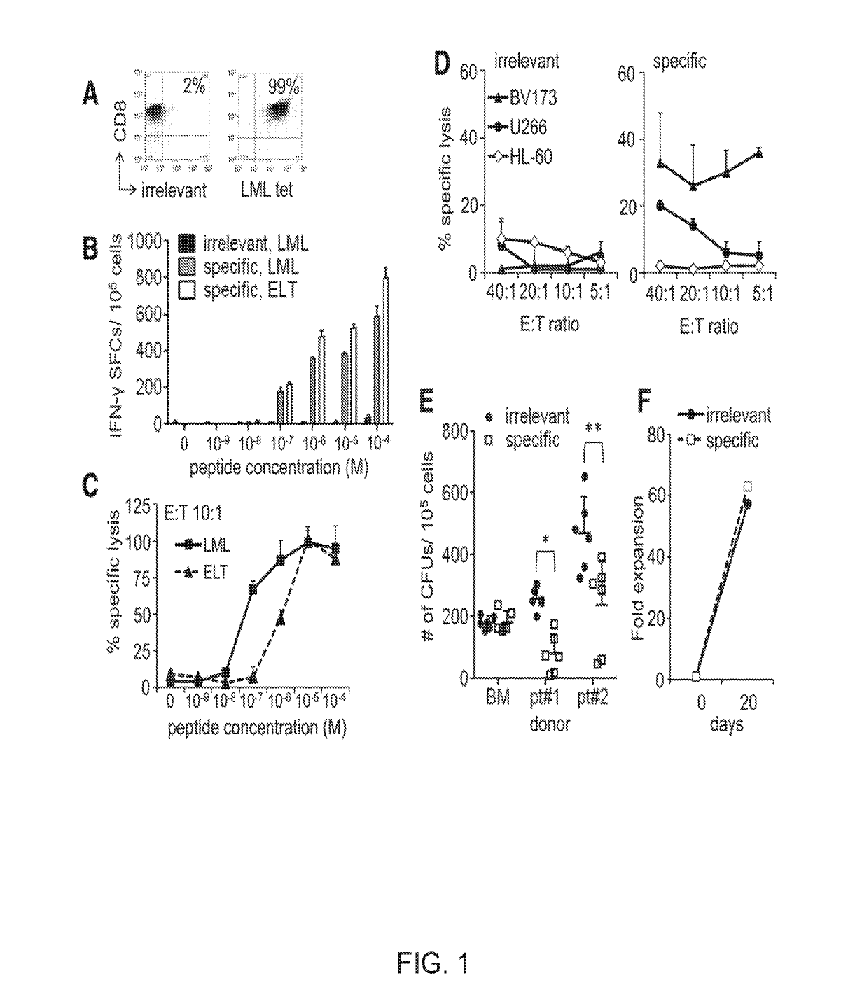

As assessed by IFN-γ ELISpot, 3 of the 5 CTLs lines (from donors #2, 4 and 5) were specifically reactive to both the LML (643±5, 49±1 and 96±7 SFCs/10⁵ T cells) and the ELT peptides (662±65, 45±6 and 86±9 SFCs/10⁵ T cells) after 3 antigen-specific stimulations. Single T-cell clones were generated by limiting dilution from the most reactive donor (#2) and, using multiple assays comparing survivin-specific and non-specific (irrelevant) clones, there was identified one with optimal functional avidity. Specifically, clone #24 showed the highest specificity for the LML-tetramer (>99%) (FIG. 1A) and highest TCR avidity for both LML and ELT peptides (10⁻⁷M when assessed by IFN-γ ELISpot assay (FIG. 1B), and 5×10⁸M for LML and 10⁻⁶M for ELT when measured by standard ⁵¹Cr-release (FIG. 1C). The functional avidity of clone #24 overlapped the broad range of avidities previously described for “fratricide” TCRs (Table 1). Importantly, clone #24 showed cytotoxic activity against the HLA-A*02⁺survivin⁺ tumor cell lines BV173 (leukemia) and U266 (myeloma) (FIG. 1D), and inhibition of colony forming units (CFU) of HLA-A*02⁺survivin⁺ leukemic progenitors (FIG. 1E). By contrast, the same clone was not cytotoxic against the HLA-A*02⁻survivin⁺ cell line HL-60 or against HLA-A*02⁺ normal hematopoietic progenitor cells (FIGS. 1D, E). This clone expanded effectively in vitro (>63-fold expansion after 3 weeks) (FIG. 1F) indicating lack of detectable T-cell fratricide effects.

TABLE 1
Functional avidities of survivin-specific T-cell
clones against LML-peptide pulsed T2 cells.

| | avidity by 4-hour ⁵¹Cr-release assay |
|---|---|
| | 50% lysis at E:T 10:1 [LML peptide, M] |
| clone | |
| #24 | 5 × 10⁻⁸ |
| Published TCRs with | |
| fratricide: | |
| A66 | 5 × 10⁻⁸ |
| A71 | 1.3 × 10⁻⁶ |
| A72 | 5 × 10⁻¹¹ |

TCRs A66, A71 and A72 are published allo-restricted survivin-specific TCRs (Leisegang, et al, 2010).

### Example 2

### Polyclonal T Cells Engineered to Express the Survivin-Specific TCR are Not Fratricidal

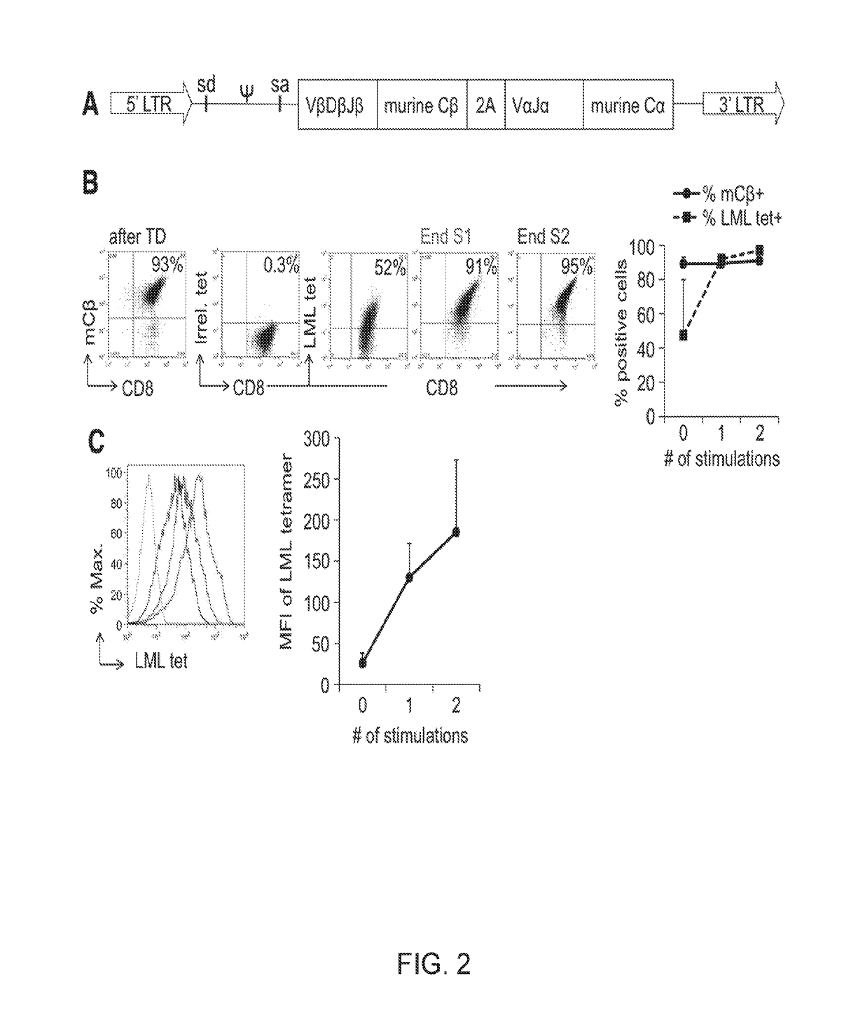

TCR α- and β-chains of clone #24 (hereafter named s24-TCR) were cloned, codon-optimized and encoded into a retroviral vector after replacement of the constant regions with the corresponding murine regions (FIG. 2A). TCR chain usage and complementarity determining regions were completely distinct from the previously published fratricide TCRs (Tables 2 and 3).

TABLE 2
Survivin-specific TCR α-chain usage.

| clone | TRAV | TRAJ | C | AA junction |
|---|---|---|---|---|
| s24 | 13-2*01 | 24*02 | A | CAETVTDSWGKLQF |
| | | | | (SEQ ID NO: 19) |

Published TCRs with fratricide:

| | | | | |
|---|---|---|---|---|
| A66 | 13-1*02 | 39*01 | A | CAARAGNMLTF |
| | | | | (SEQ ID NO: 6) |
| A71 | 12-2*01 | 31*01 | A | CAVNNARLMF |
| | | | | (SEQ ID NO: 7) |
| A72 | 14/DV4*02 | 4*01 | A | CAMREGGGYNKLIF |
| | | | | (SEQ ID NO: 8) |

Nomenclature according to the international Immunogenetics information system website www.imgt.org. Sequences of TCRs A66, A71 and A72 are published allo-restricted survivin-specific TCRs with fratricide (Leisegang, et al., 2010).

TABLE 3
Survivin-specific TCR β-chain usage.

| clone | TRBV | TRBD | TRBJ | C | AA junction |
|---|---|---|---|---|---|
| s24 | 15*02 | 1*01 | 1-5*01 | B1 | CATSRGDSTAEPQHF |
| | | | | | (SEQ ID NO: 9) |

Published TCRs with fratricide:

| | | | | | |
|---|---|---|---|---|---|
| A66 | 30*01 | 2*01 | 2-7*01 | B1 | CAWGTGLALYEQYF |
| | | | | | (SEQ ID NO: 10) |
| A71 | 30*01 | 1*01 | 2-1*01 | B1 | CAWSIGAEQFF |
| | | | | | (SEQ ID NO: 11) |
| A72 | 30*02 | 1*01 | 1-1*01 | B1 | CAGQDLNTEAFF |
| | | | | | (SEQ ID NO: 12) |

Nomenclature according to the international Immunogenetics information system website www.imgt.org Sequences of TCRs A66, A71 and A72 are published allo-restricted survivin-specific TCRs with fratricide (Leisegang, et al., 2010).

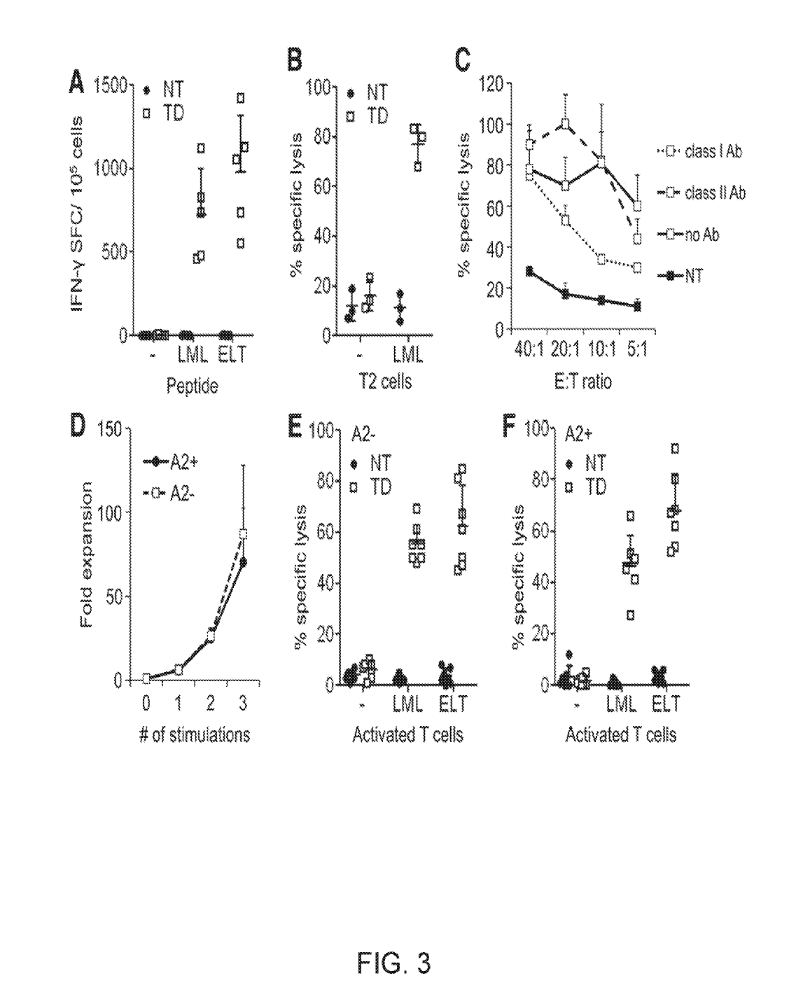

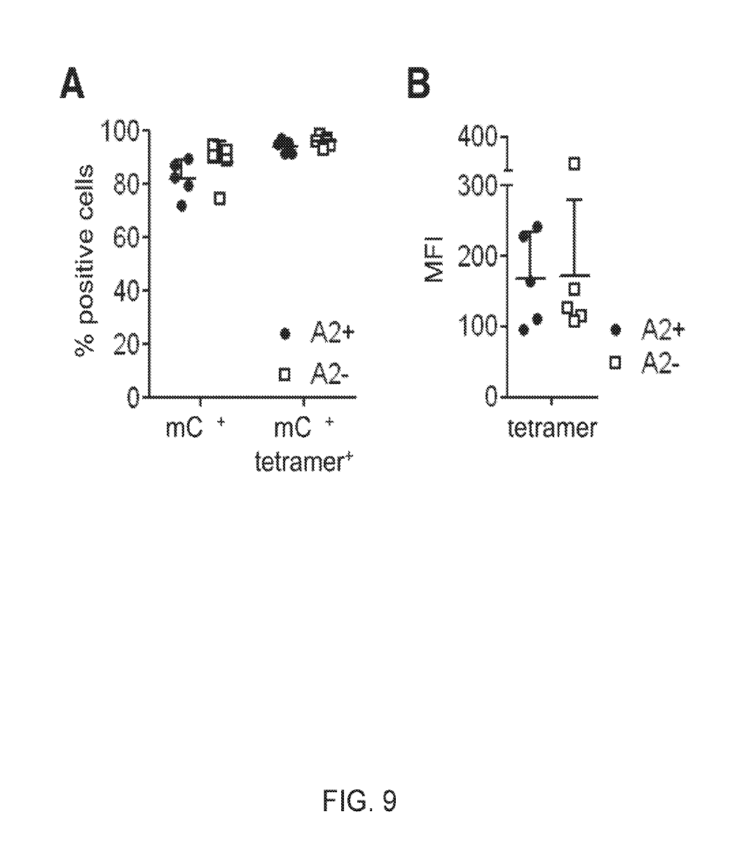

CD8+ T cells were transduced and expanded in the presence of LML-peptide pulsed artificial APCs (aAPCs) and IL-2. Immediately after transduction, 89%±4% of T cells stained for the murine constant β-chain (mCβ), and 47%±32% with the LML-tetramer (FIG. 2B). Although positivity for the LML-tetramer was modest, with a mean fluorescence intensity (MFI) of 26±12, after expansion in the presence of LML-pulsed aAPCs, there was a significant enrichment in LML-tetramer cells (97%±1%) (FIGS. 2B, C). The ectopically expressed s24-TCR was functional with s24-TCR⁺ T cells producing IFN-γ in response to both the LML (725±274 SFCs/10⁵ T cells) and the ELT (978±341 SFCs/10⁵ T cells) peptides (FIG. 3A). s24-TCR⁺ T cells also lysed LML-peptide pulsed T2 cells (77%±8% specific lysis, E:T 20:1) (FIG. 3B) in an HLA-restricted fashion, as cytotoxic activity was significantly reduced by pre-incubation with MHC class-I blocking antibodies (FIG. 3C) (53%±10% specific lysis, E:T 20:1; p=0.03). To confirm that s24-TCR⁺ T cells did not produce fratricide, phenotype, expansion and cytotoxic activity were compared of s24-TCR⁺ cells generated from both HLA-A*02 and HLA-A*02⁻ donors. The TCR was efficiently expressed in both (FIGS. 9), and s24-TCR⁺ T cells expanded identically in response to LML-pulsed aAPCs and IL-2 (66±38 vs 76±38 fold expansion after 3 stimulations for HLA-A*02⁺ and HLA-A*02⁻ donors, respectively) (FIG. 3D). Furthermore, there was no detectable cytotoxic activity by s24-TCR⁺ T cells against HLA-A*0201⁻ T cells. As shown in FIGS. 3E and F, lysis of activated T cells was negligible (2%±4% vs 6%±3% specific lysis, E:T 20:1, HLA-A*02⁺ vs HLA-A*02⁺ donors), and these cells became targetable by s24-TCR⁺ T cells only after loading with the LML or ELT peptide (46%±12% vs 55%±7% specific lysis for LML-loaded T cells; 68%±14% vs 62%±16% for ELT-loaded T cells, E:T 20:1, HLA-A*02⁺ vs HLA-A*02⁻ donors). As expected, control T cells had no cytotoxic activity against activated T cells (FIGS. 3E, F).

### Example 3

Survivin-TCR Redirected T Cells Exert Antitumor Activity Without Toxicity to Normal Hematopoietic Progenitor Cells

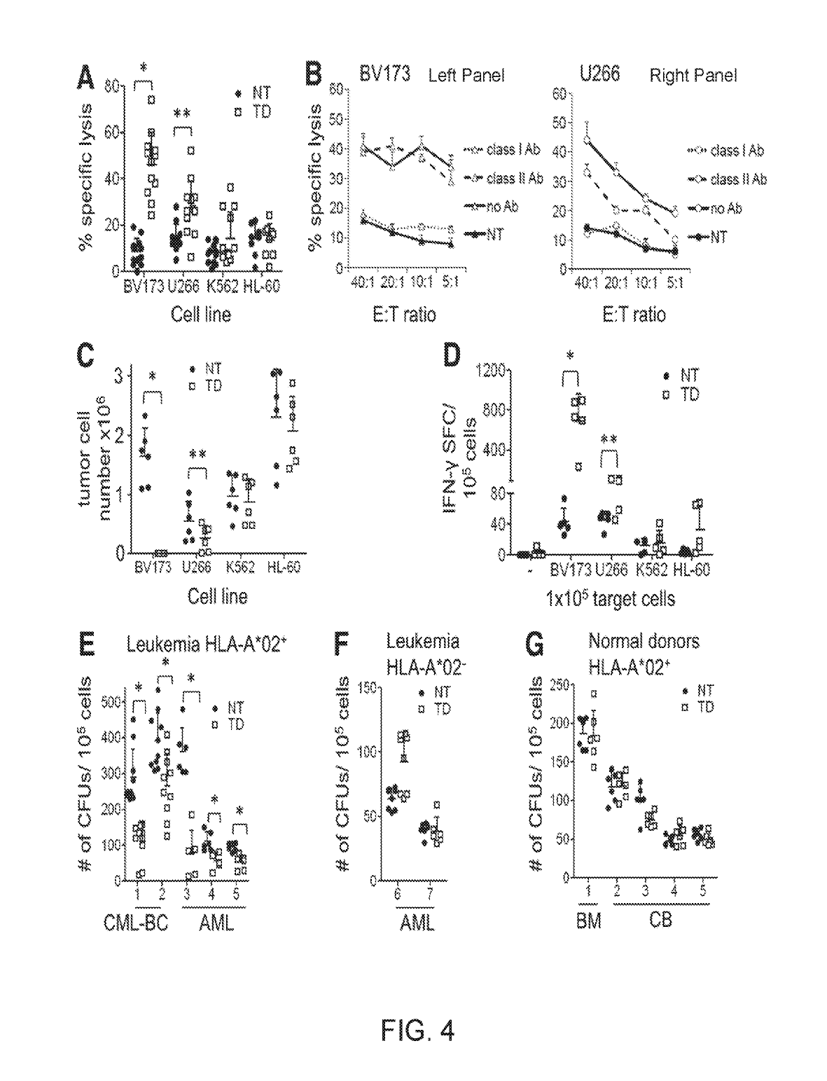

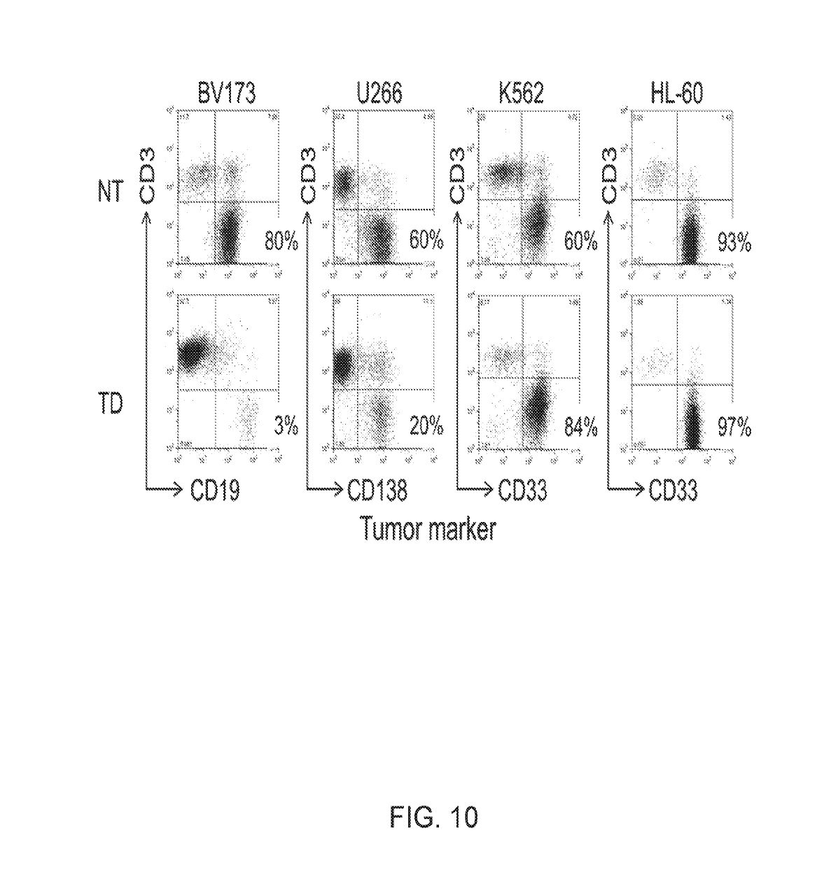

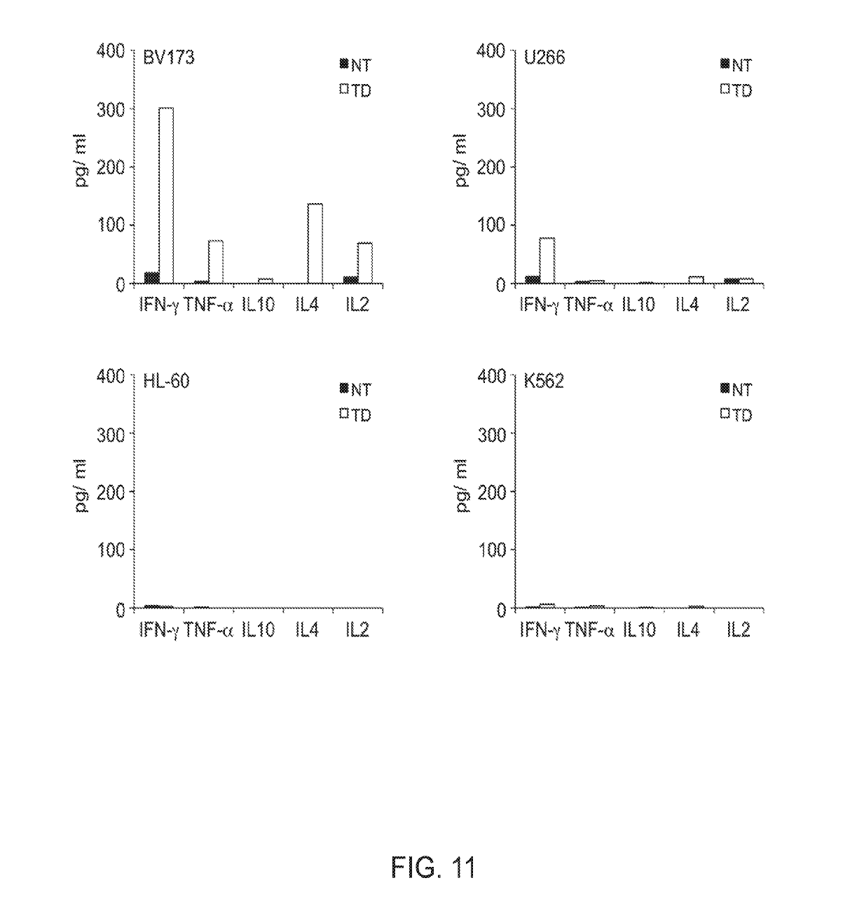

To ensure that the lack of fratricide did not occur at the expense of reduced antitumor activity, the cytotoxic activity of s24-TCR⁻ T cells against survivin⁺ hematological malignancies was evaluated. s24-TCR⁺ T cells produced significantly greater lysis of the HLA-A*02⁺survivin⁺ leukemia cell line BV173 (46%±14% specific lysis at a 20:1 E:T ratio) and the HLA-A*02⁺survivin⁺ multiple myeloma-derived cell line U266 (27%±12%) as compared to control T cells (8%±6% and 14%±6%, respectively) (p<0.001 for BV173, p=0.003 for U266) (FIG. 4A). In contrast, negligible killing was observed for both transduced and control T cells against control targets HLA-A*02⁻survivin⁺ leukemia cell lines K562 and HL-60 (FIG. 4A). Cytotoxic activity of s24-TCR⁺ T cells was MHC class-I restricted as pre-incubation of target cells with HLA class-I blocking antibodies abrogated the cytotoxic activity against BV173 and U266 cells (FIG. 4B). In longer-term assays in which there was co-cultured control or s24-TCR⁺ T cells with these HLA-A*02⁺survivin⁺ tumor cells for five days, there was significant reduction of both BV173 and U266 tumor cells only in the presence of s24-TCR⁺ T cells (FIG. 4C, FIG. 10). These cytotoxic effects were paralleled by IFN-γ production by s24-TCR⁺ T cells against the BV173 and U266 cell lines as assessed by ELISpot assays (FIG. 4D) and by release of Thl cytokines as assessed by cytometric bead arrays (FIG. 11). Antitumor effects of s24-TCR⁺ T cells were also confirmed in CFU assays against primary leukemic samples. As shown in FIG. 4E, leukemic CFU formation was significantly reduced in all five HLA-A*02⁺ leukemia samples incubated with s24-TCR⁺ T cells as compared to control T cells, with a median reduction of CFU formation of 48% in the presence of s24-TCR⁺ T cells (range 32-78%; p=0.03). In addition, no cytotoxic effects were observed against two HLA-A*0201 leukemia samples (FIG. 4F). In sharp contrast, CFU formation of hematopoietic stem/progenitor cells from HLA-A*0201⁺ healthy donors was unaffected by incubation with s24-TCR⁺ T cells, with only a median 3% reduction of CFU in the presence of s24-TCR⁺ T cells as compared to cultures with control T cells (FIG. 4G).

### Example 4

### Survivin-TCR Transgenic T Cells Have Antitumor Activity In Vivo and Improve Survival

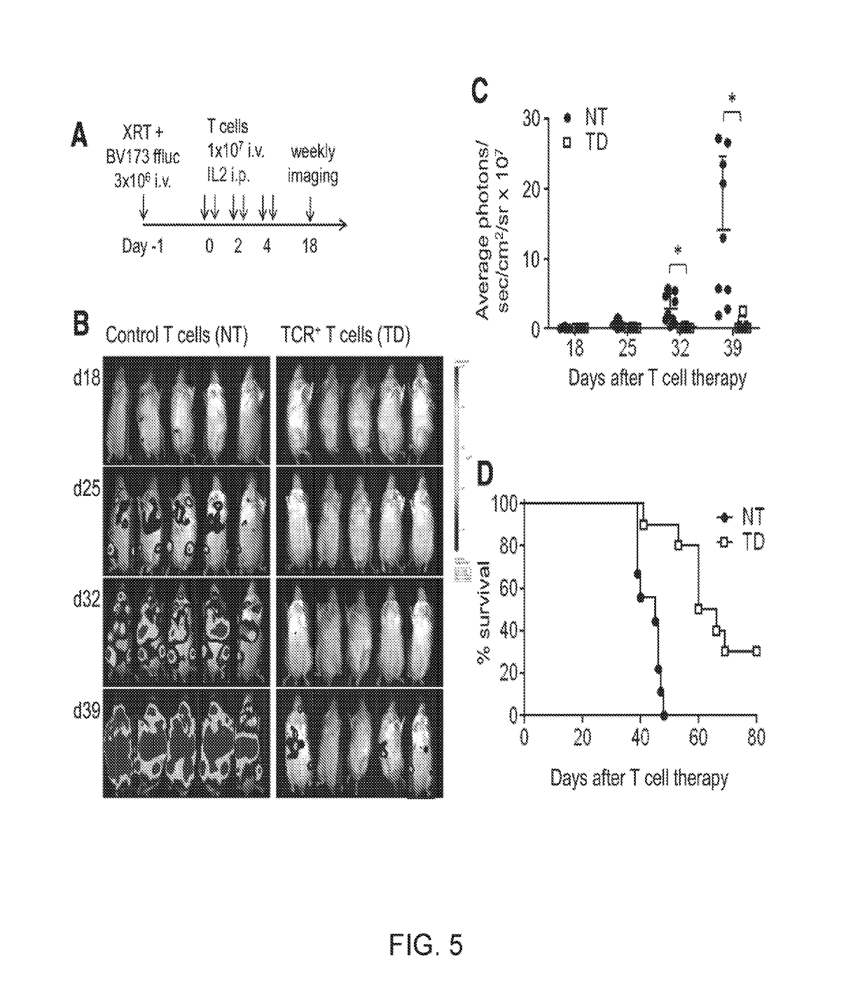

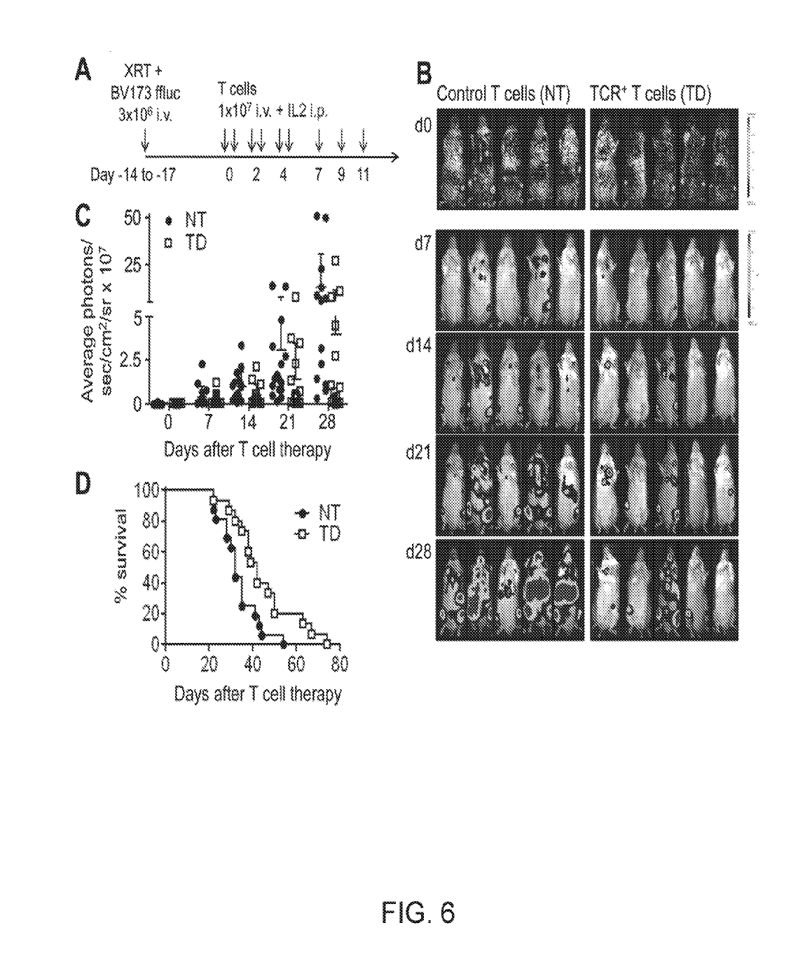

To confirm the in vivo antitumor function of s24-TCR+ T cells, a xenogeneic NSG mouse model was used that is systemically engrafted with BV173 cells genetically modified with Firefly luciferase (FFLuc) and used bioluminescent imaging (BLI) to monitor tumor growth. In conditions that mimic residual leukemia, mice received adoptive T-cell transfer with either control or s24-TCR⁺ T cells the day after leukemia infusion (FIG. 5A). At day 40 post infusion, mice treated with s24-TCR⁺ T cells had significantly better control of leukemia growth as compared to mice receiving control T cells (8.1×10⁶±9×10⁶ vs. 195×10⁶±85×10⁶ photons/sec) (p=0.003) (FIGS. 5B, C). This translated into an improved overall-survival of s24-TCR⁺ treated mice by day 80 (p<0.001) (FIG. 5D), with 3/10 s24-TCR⁺ T-cell treated mice tumor free. To measure antitumor activity in mice with high leukemic burden, T cells were infused 2 weeks after leukemia inoculation when disease dissemination and burden was documented by BLI (FIG. 6A). Mice receiving s24-TCR⁺ T cells had a significantly slower leukemia progression as compared to control mice, resulting in a lower bioluminescent signal by day 28 (FIGS. 6B, C) (40×10⁶±71×10⁶ vs. 128×10⁶±176×10⁶ photons/sec) (p=0.04). This TCR-mediated anti-leukemic activity translated to significantly improved survival of the mice (p=0.01) (FIG. 6D).

### Example 5

### Alanine-Scanning Reveals a TCR Binding Mode Optimized for Recognition of the Survivin Epitope

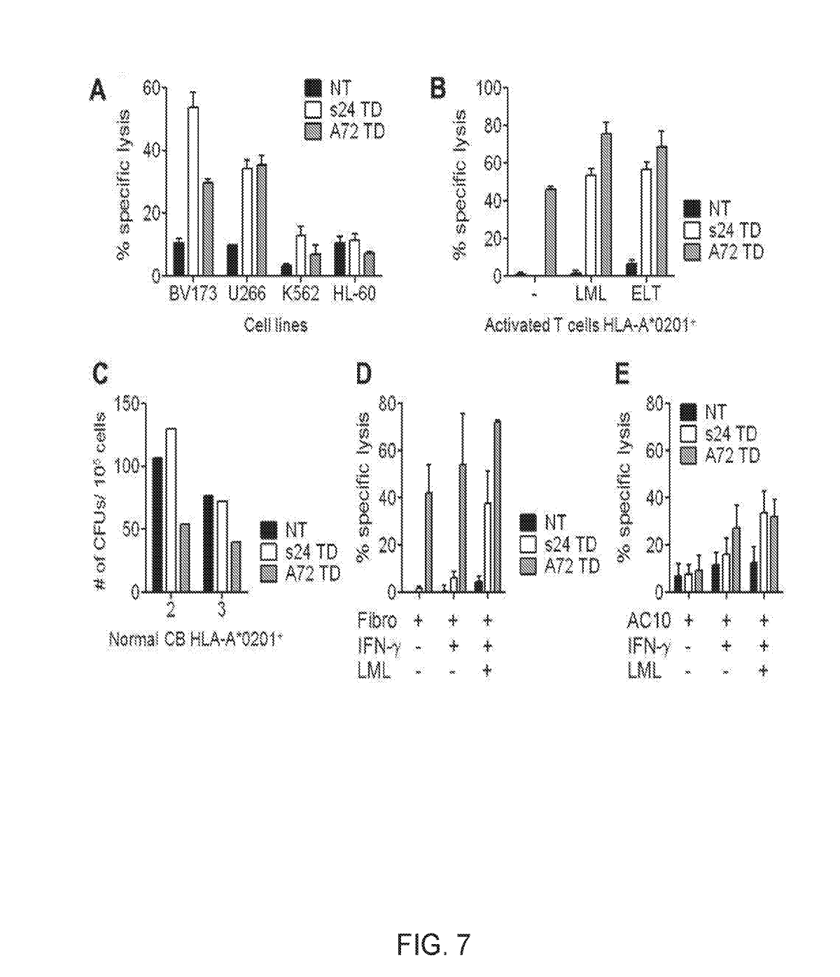

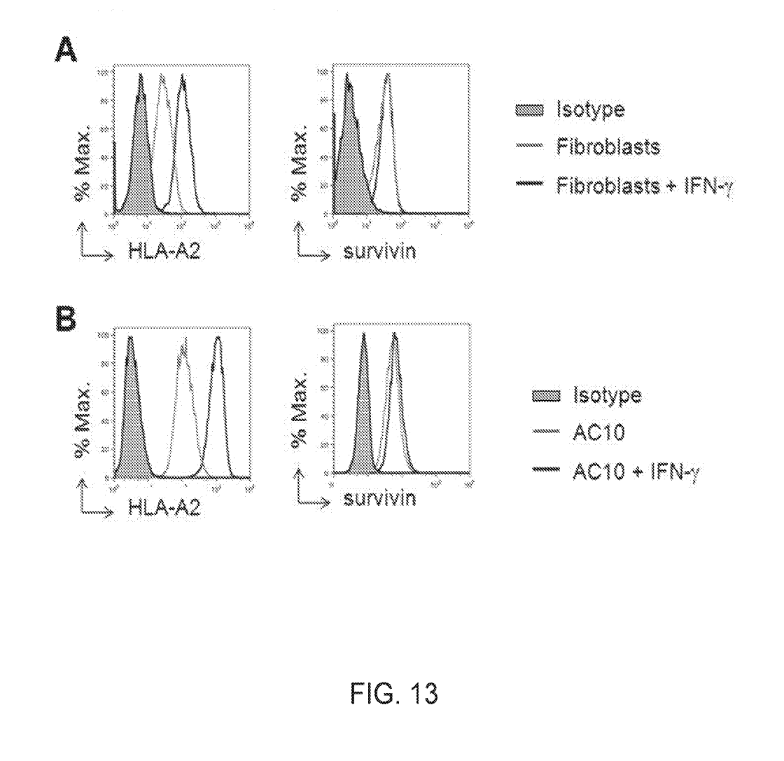

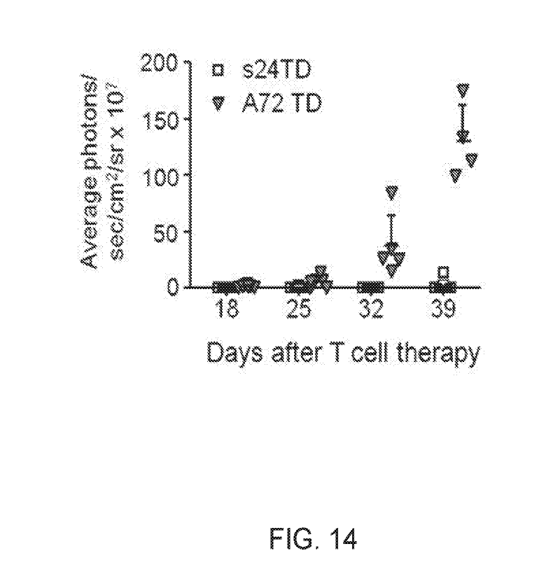

To understand the mechanism by which the s24-TCR produced antitumor activity without fratricide or toxicity to normal hematopoietic cells, T cells expressing either s24-TCR or the reported “fratricide” TCR (A72-TCR) (Leisegang, et al., 2010) were compared in side by side experiments. While no significant differences were observed between T cells expressing either TCR in terms of antitumor activity in vitro (FIG. 7A), only T cells expressing A72-TCR showed autoreactivity (FIG. 7B) and toxicity against normal hematopoietic stem/progenitor cells (FIG. 7C). Furthermore, A72-TCR+ T cells but not s24-TCR+ cells also showed cytotoxic activity against non-hematopoietic cells such as fibroblasts (FIG. 7D) and cardiomyocytes (FIG. 7E). Importantly, the safer profile by s24-TCR+ T cells was retained even in conditions mimicking an inflammatory insult, such as when targets were preincubated with IFN-γ which modulates HLA-A*0201 expression (FIG. 13). This favorable toxicity profile was not due to a reduced antitumor activity in vivo in the BV173 tumor model, as s24-TCR+ T cells mediated superior tumor control compared to A72-TCR+ T cells (p<0.0001) (FIG. 14).

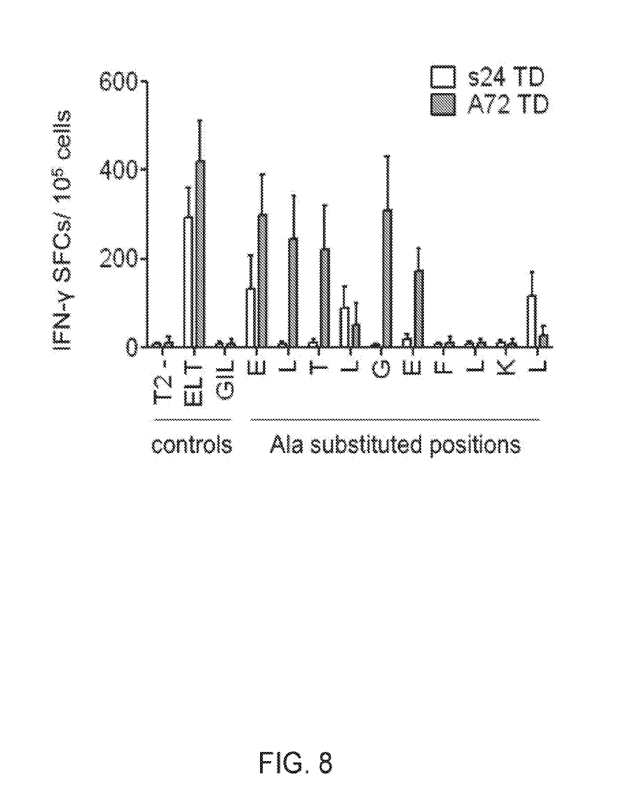

Computer modeling studies showed that the selective tumor specificity of the s24-TCR relies on a tight and extended interface of the TCR with the survivin-MHC complex, in contrast to the A72-TCR that interacted mostly with the HLA-A*02 groove. Specifically, s24-TCR created a network of highly-optimized physical interactions involving numerous aromatic residues with the local region of the survivin peptide including Leu4, Gly5 and Phe7. The structural analysis was then corroborated by functional analyses of the TCR-peptide-MHC interaction were performed by alanine-substitution experiments of the survivin peptide. As shown in FIG. 8, every single residue (10/10) of the survivin peptide appeared to be crucial for s24-TCR functional activation because 7/10 single substitutions completely abrogated IFN-γ release and 3/10 significantly reduced it. By contrast, only 3/10 substitutions were critical for the complete functional loss of the A72-TCR activation (FIG. 8), suggesting a smaller and less optimal TCR-peptide binding mode. Based on both the prediction model and the alanine substitution analysis, the UniProtKB/Swiss-Prot database sequence was queried for protein sequences containing the motifs, XLTXGEFLKX (SEQ ID NO:13) and the XXXLXXFLKL (SEQ ID NO:14) to identify potential cross-reactive epitopes. Seven peptides were selected that were associated with cells of the hematopoietic or immune system, including T lymphocytes. IFN-γ ELISpot assays showed that A72-TCR, but not s24-TCR, reacted against T2 cells pulsed with several of these peptides (Table 4).

TABLE 4
Different molecular recognition patterns of autologous versus
allogeneic repertoire derived survivin-TCRs.

| | | SEQ | | | Reactive^{A} | |
|---|---|---|---|---|---|---|
| | Peptide sequence, conserved residues | ID | | Abbrevi- | TCR | |
| # | (underlined) | NO: | Antigen | ation | s24 | A72 |
| | E L T L G E F L K L | 20 | Survivin | ELT | Yes | Yes |
| 1 | 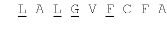 | 21 | CD3d | LAL | No | Yes |
| 2 | 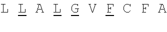 | 22 | CD3d | LLA | No | No |
| 3 | 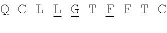 | 23 | CD81 | QCL | No | Yes |
| 4 | 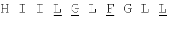 | 24 | CSF3R | HII | No | No |
| 5 | 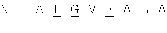 | 25 | CRLS1 | NIA | No | No |
| 6 | 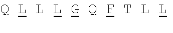 | 26 | EPB42 | QLL | No | No |
| 7 | 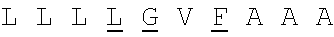 | 27 | INGR2 | LLL | No | No |

^{A}Epitopes predicted by alanine-substitution analyses were loaded on T2 cells and reactivity by s24-TCR⁺ or A72-TCR⁺ T cells assessed by IFN-γ ELISpot assays. Representative results of 3 donors. Abbreviations: CD3d: CD3 delta; CD81: CD81 antigen; CSF3R: Granulocyte colony stimulating factor receptor; CRLS1: cardiolipin synthase; EPB42: Erythrocyte membrane protein band 4.2.; INGR2: Interferon gamma receptor 2.

### Example 6

### Summary of Certain Embodiments

The disclosure demonstrates isolation from an autologous TCR repertoire of a novel survivin-specific s24-TCR that when engrafted in polyclonal T cells shows sufficient functional avidity to eliminate a variety of tumor cells both in vitro and in vivo without producing auto-toxicities. This novel TCR is capable of discriminating survivin on self-tissues from tumor-associated survivin expression and selectively mediates antitumor reactivity without “on-target off-tumor” toxicity. Functional data reveal that the selective tumor specificity of the s24-TCR relies on a highly specific interaction of the TCR with the survivin-MHC complex. Thus, the optimal recognition of a self peptide by the TCR confers its selectivity, minimizing cross-reactivity and hence auto-reactivity. The findings challenge the previous conclusion that functional survivin-specific TCRs are toxic, and therefore not suitable for clinical use, and the claim that the fratricide effect mediated by such TCRs are exclusively due to “on-target” recognition of activated T cells (Leisegang, et al., 2010). The observation was generalized in a comparative analysis on a set of seven additional TCRs derived from autologous or allogeneic repertoires targeting several different TAAs, suggesting that the thymic selection step is key for minimizing TCR cross-reactivity.

Numerous studies have indicated that survivin is upregulated in most cancers, but it is also functional in normal cells such as CD34⁺ hematopoietic stem cells, T lymphocytes (Alfieri, 2003) and cardiomyocytes (Wohlschlaeger, et al., 2010; Levkau, 2011). For instance, conditional knockout mice show that loss of survivin at early stage blocks T-cell transition from the double-negative to double-positive cells while at late stages decreases their number in the circulation (Xing, et al., 2004). Survivin was also found upregulated upon TCR crosslinking in human T cells (Leisegang, et al., 2010; Kornacker, et al., 2001), and is involved in proliferation of cardiac myocyte remodeling during congestive cardiac failure (Wohlschlaeger, et al., 2010) and in the ischemic/reperfused heart (Levkau, 2011). Despite these functional activities of survivin, no cardiac toxicities and no delays in stem cell engraftment or T-cell reconstitution have been reported in patients receiving survivin-based vaccines after autologous stem cell transplantation even with elicitation of survivin-specific CTLs (Rapoport, et al., 2011). In line with this clinical experience, there was no impairment in the growth of normal hematopoietic progenitors, or any negative impact on T-cell expansion in the presence of the s24-TCR redirected T cells. This safety profile occurred without compromising antitumor effects as leukemic progenitors and tumor cells were significantly eliminated both in vitro and in vivo by polyclonal T cells expressing the s24-TCR. These data thus suggest that the s24-TCR recognizes tumor targets, but is “tolerant” to healthy cells that express the antigen below the threshold of recognition. By contrast, toxic effects were consistently induced by polyclonal T cells expressing the A72-TCR that was isolated from allogeneic HLA-mismatched TCR repertoires.

The conclusion that s24-TCR selectively recognizes tumor cells is supported at least by functional alanine substitution analyses of the survivin epitope. These studies demonstrate that the s24-TCR is not “fratricidal” or auto-reactive because it establishes most of its strongest interactions with the survivin peptide, while the known “fratricidal” A72-TCR favors peptide cross-reactivity. The findings are in line with previous studies showing that native cross-reactivity appears to be focused on a limited number of hot spot residues in any given peptide-MHC complex (Tynan, et al., 2005; Birnbaum, et al., 2014). Murine studies previously showed that peptide cross-reactivity only occurs in an allogeneic setting as negative selection occurring in physiological conditions severely limits the number of distinct ligands recognized by a TCR (Huseby, et al., 2003). Cross-reactive TCRs can be found only in mice in which negative selection has been experimentally limited and thus are “accepting” amino-acid substitutions within the targeted peptide (Huseby, et al., 2006). By comparing the molecular recognition patterns of the autologous versus allorestricted survivin-TCRs, there was identified the molecular determinants explaining the mechanism for the capability of the s24-TCR to discriminate native from tumor survivin expression levels. It was therefore considered that the “fratricidal” effect reported with the survivin specific A72-TCR generated from an allogeneic TCR repertoire may be due not only to a lower threshold of “on-target” recognition on activated T cells but also to an “off-target” recognition of cross-reactive peptides due to a suboptimal peptide-TCR interaction. As a consequence, the in vivo anti-tumor function of A72-TCR+ T cells may be limited in comparison to the s24 TCR, as it was indeed observed in the BV173 mouse model. By validating the findings with an additional set of autologous and allogeneic TCRs targeting different TAAs, the study suggests that cross-reactivity with potential for “off-target” toxicity may be more a general problem of TCRs isolated from allogeneic repertoires. The findings have broader implications for therapeutic tumor targeting by means of transgenic TCRs. While TCRs derived from allogeneic or xenogenic repertoire, or with experimentally enhanced affinities have been widely used as means to attain high-affinity TCRs, autologous repertoires, still remain a valid strategy.

In conclusion, the disclosure re-establishes the validity of survivin as a target in cancer immunotherapy by means of the ectopic expression of a TCR that fulfills the requirements of epitope specificity, antitumor activity and lack of autoreactivity. This TCR relies on the optimal and selective recognition of the MHC-epitope complex and is capable of sensing survivin antigen levels on self-tissue versus tumor targets. This approach is adaptable for the identification of additional TCRs targeting other shared tumor/self-antigens, reducing the risk of generating TCR-mediated autoreactivity.

### Example 7

### Exemplary Methods

Cell Lines

The tumor cell line BV173 (B-acute lymphoblastic leukemia) was obtained from the German Cell Culture Collection (DSMZ, Braunschweig, Germany), and the tumor cell lines U266B1 (multiple myeloma), K562 (erythroleukemia), HL-60 (acute myelomonocytic leukemia), CEM-T2 (TAP transporter deficient), 293T and the cardiomyocyte cell line AC10 from the American Tissue Culture Collection (ATCC, Manassas, Va.). Cells were maintained in culture with RPMI 1640 medium (HyClone, Thermo Scientific, Waltham, Mass.) or IMDM medium (Gibco, Invitrogen Life Technologies, Grand Island, N.Y.) for 293T cells, or DMEM/F12 medium (Gibco) for AC10 cells, containing 10% or 20% fetal bovine serum (FBS, HyClone) as per suppliers recommendations, 1% L-glutamine and 1% penicillin/streptomycin (Invitrogen) in a humidified atmosphere containing 5% CO₂ at 37° C. The BV173 cell line was transduced with a retroviral vector encoding the Firefly-luciferase (FFluc) and neomycin resistance genes as previously described (Hoyos, et al., 2010). The K562 cell line was engineered to express the HLA-A*0201 molecule and CD4OL, CD80 and OX4OL as co-stimulatory molecules, and used as artificial antigen presenting cells (aAPCs) for T cell expansion (Quintarelli, et al., 2008). Cell lines were authenticated by the University of Texas MD Anderson Cancer Center Characterized Cell Line Core Facility. AC10 cells were confirmed to be HLA-A*0201⁺ by high resolution Sequence Based Typing (Houston Methodist Hospital, Houston, Tex.).

Samples from Healthy Donors and Leukemia Patients

Buffy coats from healthy volunteer blood donors were obtained through the Gulf Coast Regional Blood Center, Houston, Tex. Deidentified cord blood (CB) units were obtained through the MD Anderson Cord Blood Bank (University of Texas, Houston, Tex.) on an IRB-approved protocol. Peripheral blood (PB) and bone marrow (BM) samples from de-identified patients with AML or CML were collected according to local institutional review board (IRB)-approved protocols (Baylor College of Medicine, Houston, Tex.) or provided by the Texas Children's Cancer Center Tissue Bank. Dermal fibroblasts were collected from HLA-A*0201+ healthy donors (confirmed by high resolution Sequence Based Typing, Houston Methodist Hospital, Houston, Tex.) according to the local BCM-IRB-approved protocol and generated as previously reported (Leen, et al., 2004).

Peptides and Alanine Substitution Experiments

The native 10-mer peptide survivin95-104 ELTLGEFLKL (ELT; SEQ ID NO:15), its heteroclitic 9-mer variant survivin₉₆₋₁₀₄97M LMLGEFLKL (LML; SEQ ID NO:16) , Preferentially Antigen Expressed in Melanoma (PRAME) P435 (P435, NLTHVLYPV [SEQ ID NO: 29]), PRAME P300 (P300, ALYVDSLFFL [ SEQ ID NO: 30]), MART-1 ELA (ELAGIGILTV [SEQ ID NO: 31]), tyrosinase YMD (YMDGTMSQV [SEQ ID NO: 32]) and alanine substitution variants for each amino acid position of all peptides were synthesized by Genemed Synthesis (San Antonio, Tex.). All peptides were reconstituted in DMSO and used at a concentration of 5μM unless otherwise indicated. Preferentially Antigen Expressed in Melanoma (PRAME) P435 peptide (P435, NLTHVLYPV; SEQ ID NO:17) or influenza matrix protein₅₈-₆₆ (flu, GILGFVFTL; SEQ ID NO:18) were used as irrelevant controls (Quintarelli, et al., 2008). Recognition of the HLA-peptide complex by transgenic T cells was analyzed by IFN-γ ELISpot assay using peptide pulsed T2 cells as targets.

Generation and Expansion of Survivin-Specific T-Cell Lines and Clones

Peripheral blood mononuclear cells (PBMCs) were isolated by Lymphoprep (Accurate Chemical and Scientific Corp., Westbury, N.Y.) density gradient centrifugation. HLA-A2 status was assessed by flow cytometry (FACS) and survivin-specific T-cell lines were generated from HLA-A2 positive donors as previously described (Quintarelli, et al., 2008). Briefly, dendritic cells (DCs) were generated from CD14-selected monocytes (using CD14⁺ beads and manual MACS columns, Miltenyi Biotech, Auburn, Calif.) and, after maturation, pulsed with 5 μM of the specific peptide for 2 hours at 37° C. DCs were then used to stimulate autologous CD8⁻ T cells (obtained by immunomagnetic selection, Miltenyi Biotech) at an effector to target (E:T) ratio of 20:1 in complete CTL media (45% Click's media (Irvine Scientific, Santa Ana, Calif.), 45% RPMI 1640, 5% heat-inactivated human AB serum (Valley Biomedical, Winchester, Va.), 1% L-glutamine and 1% penicillin/streptomycin (Invitrogen, Carlsbad, Calif.), in the presence of a previously validated combination of cytokines (IL-7 (long/m1), IL-12 (lng/m1), and IL-15 (2ng/m1) (from Peprotec, Rocky Hill, N.J. or R&D Systems, Minneapolis, Minn.). At day 9 and 16 of culture, T cells were re-stimulated with peptide-pulsed artificial antigen presenting cells (aAPCs) at an E:T ratio of 10:1 in media containing IL-7, IL-12, and IL-15. Interleukin-2 (50 U/ml) (Teceleukin, Hoffmann La-Roche, Nutley, N.J.) was added to the culture from day 16, as previously described (Quintarelli, et al., 2008).

Single cell survivin-specific T-cell clones were generated from LML and ELT reactive T-cell lines by limiting dilution as previously described (Perna, et al., 2013). Growing cells were screened for survivin-specific reactivity in IFN-γ ELISpot assays and were further expanded in the presence of allogeneic feeder cells, IL-2 and OKT3 (Orthoclone). In parallel, non-specific (irrelevant) clones were expanded from the same donors and served as controls. The expanded clones were confirmed to be HLA-A*0201+ by high resolution Sequence Based Typing (The Methodist Hospital, Houston, Tex.). PRAME-specific clones were generated following the same methodology.

Immunophenotyping

Cells were stained with fluorescein isothiocyanate (FITC)-, phycoerythrin (PE)-, peridinin chlorophyll protein (PerCP)- or allophycocyanin (APC)-conjugated antibodies for HLA-A2, CD3, CD4, CD8, CD33, CD34, CD38, CD56, CD45 from BD Biosciences (San Jose, Calif.) or Beckman Coulter (Brea, Calif.), PE-conjugated antibody for survivin (R&D Systems), APC-conjugated antibody for murine TCR constant β-chain (eBioscience, San Diego, Calif.), or PE-conjugated LML or ELT survivin specific tetramers prepared by the Baylor College of Medicine MHC Tetramer Production Facility. Data acquisition was performed on a FACS Calibur using CellQuest software (BD). Data analysis was performed using FlowJo Software (Treestar, Ashland, Oreg.).

ELISpot Assay

The Interferon-γ (IFN-γ) ELISpot assay was performed as previously described (Quintarelli, et al., 2008). In brief, 1×10⁵ T cells/well were plated in triplicates and then stimulated with 5 μM or the indicated concentration of the specific peptides, or 1×10⁵ cells of the respective target cell lines or media alone. As positive control, T cells were stimulated with 25 ng/ml of Phorbol myristate acetate (PMA) (Sigma-Aldrich, St. Louis, Mo.) and 1 μg/ml of ionomycin (Sigma-Aldrich). The IFN-γ spot forming cells (SFCs) were enumerated (ZellNet, Fort Lee, N.J.).

Chromium-Release Assay

The cytotoxic activity of T cells was evaluated using a standard 4-hour (for determination of TCR avidity using peptide-pulsed T2 cells) to 6-hour (for assessing killing of tumor cell lines, activated T cells, fibroblasts and cardiomyocytes) ⁵¹Cr-release assay as previously described (Quintarelli, et al., 2008). Target cells were incubated in medium alone or in 1% Triton X-100 (Sigma-Aldrich) to determine spontaneous and maximum ⁵¹Cr-release, respectively. The mean percentage of specific lysis of triplicate wells was calculated as follows: [(test counts−spontaneous counts)/(maximum counts−spontaneous counts)]×100%. For blocking experiments, target cells were pre-incubated with anti-HLA class I or class II blocking antibodies (DAKO, Carpinteria, Calif.) as previously described (Quintarelli, et al., 2008). In selected experiments, fibroblasts and cardiomyocytes were preincubated with IFN-γ (100 U/m1) (Peprotech) for 48 hrs before being used as targets in the absence or in the presence of the LML peptide (28).

Co-Cultures and Cytometric Bead Array

Transduced or non-transduced T cells (1×10⁶/well) were co-cultured with tumor cell lines (2×10⁵/well) at an effector to target (E:T) ratio of 5:1 in 24-well plates, in the absence of cytokines. After day 5 of culture, cells were harvested and stained for CD3 and specific tumor markers (CD19 for BV173, CD138 for U266, CD33 for HL-60 and K562). Residual tumor cells in cultures were enumerated by FACS using CountBrigth beads (Invitrogen). Co-culture supernatant was harvested after 24 hours of culture and cytokines measured using specific cytometric bead arrays (BD) according to manufacturer's instructions.

Colony Forming Unit Assay (CFU) of Leukemic and Normal Hematopoietic Progenitors

Mononuclear cells (MNCs) from BM, CB or PB of healthy donors or leukemia patients were co-incubated with survivin-specific or non-specific T-cell clones, or survivin-TCR transduced or non-transduced T cells at an effector to target (E:T) ratio of 10:1 for 6 hours and then plated in duplicate in methylcellulose-based medium supplemented with recombinant cytokines (Methocult H4434 classic, Stem Cell Technologies, Tukwila, Wash.), as previously described (Quintarelli, et al., 2008). Granulocyte-macrophage colony-forming units and erythrocyte CFUs were scored using a high-quality inverted microscope after 2 weeks of culture.

Isolation of Survivin-Specific TCR Genes and Generation of a Retroviral Vector

Total RNA was isolated from the survivin-specific clones #24 (s24), #16 (s16) or from PRAME-specific clones p11, p28 and p300 using the RNeasy kit from Qiagen (Germantown, Md.) and TCR cDNAs cloned by 5′ RACE PCR (Generacer Kit, Invitrogen) following the manufacturer's instructions. The PCR products were cloned into the pCR4 TOPO vector (Invitrogen) and transformed into One Shot TOP10 competent cells (Invitrogen). Plasmid DNAs were prepared from 40 individual colonies, 20 containing the TCR a-chain cDNA and 20 containing the TCR β-chain cDNA. Full-length inserts from 10 plasmids per TCR chain were sequenced (Seqwright, Houston, Tex.) to determine the TCR usage of the CTL clone s24. After identification, TCR sequences were modified by replacing the human with murine TCR constant regions, linked by a 2A sequence, codon-optimized by Geneart (Invitrogen) and finally introduced into the SFG retroviral vector (FIG. 2A). Native α- and β-chain TCR sequences of the A72-TCR (Leisegang, et al., 2010) were codon-optimized and synthesized by Geneart (Invitrogen) and introduced separately into the SFG retroviral vector without further modification. MART-1 (M1-29 and M1-67) and tyrosinase (T58) TCR sequences (Wilde, et al., 2009) were codon-optimized, linked by a 2A sequence, synthesized by Geneart, and introduced into the SFG retroviral vector.

Generation of Retroviral Supernatant, T-Cell Transduction and Expansion

Transient retroviral supernatant was prepared by transfection of 293T cells as previously described (Quintarelli, et al., 2007) and used to transduce CD8⁺ T cells isolated from PBMCs of healthy donors using magnetic beads (Miltenyi Biotech). Transduced cells were expanded in CTL media containing 10% FBS and weekly stimulations with survivin LML peptide-loaded y-irradiated (80Gy) aAPCs at an E:T ratio of 4:1 and IL-2 (50 U/m1). Non-transduced T cells were maintained in CTL media containing 10% FBS and IL2 (50 U/m1) and restimulated with immobilized OKT3 and anti-CD28 (BD) antibodies.

BV173 Leukemia Xenograft Model and In Vivo Bioluminescent Imaging

NSG mice (8-10 wks old) were purchased from the Jackson laboratory (Bar Harbour, Me.) and maintained at Baylor College of Medicine Animal Facility on an IACUC approved protocol. Sublethally irradiated (120 cGy) NSG mice were infused i.v. via tail vein with 3×10⁶ FFluc labeled BV173 cells. Leukemia burden was monitored by bioluminescent imaging (photons/second/cm2/sr) using the IVIS system (Xenogen, Caliper Life Sciences, Alameda, Calif.). A total of 3 T-cell infusions (2 days apart) of transduced or non-transduced T cells (10×10⁶/mouse) were injected retroorbitally either 24 hours (low tumor burden model; FIG. 5A) or 14-17 days (high tumor burden model; FIG. 6A) post BV173 inoculation. Recombinant human IL-2 (1000 U/mouse) was administered i.p. during the T-cell infusions and the following week, for a total of 6 doses. Leukemia growth was monitored weekly by imaging and survival recorded. Sick mice were sacrificed and organs (spleen, blood, bone marrow, lymph nodes, liver) were analyzed by FACS for presence of leukemia and T cells.

Expasy Prosite Motif Search for Cross-Reactive Peptides

The Expasy-PROSITE webserver (http://prosite.expasy.org/scanprosit/) was used to search for peptide motifs within all UniProtKB/Swiss-Prot data entries (release 2013_10 of Oct. 13, 2016: 541561 entries) for comparison of s24 and A72 TCRs, release 2014_07 of Jul. 14, 2009 with 546000 entries, for all TCRs). Filters were set for Homo sapiens and hematopoietic system.

Statistical Analysis

Data were summarized by means and standard deviations (SD). Student's t-test was used to determine the statistically significant differences between treatment groups with multiple comparisons adjusted by the Bonferroni method when appropriate. To compare leukemia growth trend in mice over time, bioluminescent signal intensity at every time point was log-transformed and analyzed by the robust generalized estimating equations for repeated measurements. Survival analysis was performed using the Kaplan-Meier method in GraphPad Software (La Jolla, Calif.). The log-rank test was used to assess statistical significant differences between groups of mice, with a P-value<0.05 indicating a significant difference.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Altieri, Nat Rev Cancer 3:46-54, 2003.

Andersen, et al., Cancer Res 61:5964-5968, 2001.

Barth, et al., PNAS USA. 106:1409-14, 2009.

Becker, et al., Cancer Immunol Immunother 61:2091-2103, 2012.

Birnbaum, et al., Cell. 157:1073-87, 2014.

Cameron, et al., Sci Trans' Med 5:197ra103, 2013.

Chaudhury, et al., PLoS One 6:e22477, 2011.

Cheever, et al., Clin Cancer Res 15:5323-5337, 2009.

Hoyos, et al., Leukemia 24:1160-1170, 2010.

Huseby, et al., Nat Immunol 7:1191-1199, 2006.

Huseby, et al., Proc Natl Acad Sci USA 100:11565-11570, 2003.

Johnson, et al., Blood 114:535-546, 2009.

Kornacker, et al., Immunol Lett 76:169-173, 2001.

Leen, et al., Blod. 103:1011-19, 2004.

Leisegang, et al., J Clin Invest 120:3869-3877, 2010.

Levkau, J Mol Cell Cardiol. 50:6-8m 2011.

Li, et al., Nat Biotechnol 23:349-354, 2005.

Linette, et al., Blood 122:863-871, 2013.

London, et al., Nucleic Acids Res. 39:W249-53, 2011.

Morgan, et al., Science 314:126-129, 2006.

Perna, et al., Clin Cancer Res 19:106-117, 2013.

Qian, et al., Nature 450:259-264, 2007.

Quintarelli, et al., Blood 110:2793-2802, 2007.

Quintarelli, et al., Blood 112:1876-1885, 2008.

Rapoport, et al., Blood 117:788-797, 2011.

Raveh, et al., Proteins 78:2029-2040, 2010.

Robbins, et al., J Clin Oncol 29:917-924, 2011.

Rudolph, et al., Annu Rex Immunol. 24:419-66, 2006.

Soding, et al., Nucl Acids Res. 33:W244-8, 2005.

Spranger, et al., Blood 119:3440-3449, 2012.

Tynan, et al., Nat Immunol. 6:1114-22, 2005.

Wilde, et al., Blood. 114:2131-9, 2009.

Wohlschlaeger, et al., J Heart Lung Transplant. 29:1286-92, 2010.

Xing, et al., J Exp Med 199:69-80, 2004.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1.-40. (canceled)

41. A composition comprising a genetically engineered immune cell expressing a survivin-specific T cell receptor that has antitumor activity, but lacks fratricidal effects.

42. The composition of claim 41, wherein the T cell receptor recognizes an epitope having a sequence selected from the group consisting of SEQ ID NO: 15, a functional fragment or derivative thereof having 99% identity to SEQ ID NO: 15 or SEQ ID NO: 16, and a functional fragment or derivative thereof having 99% identity to SEQ ID NO: 16.

43. A composition comprising an immune cell according to claim 41, wherein the cell comprises a survivin-specific T cell receptor which comprises one or both of the following:
(a) an alpha chain comprising SEQ ID NO: 1 or a functional fragment or derivative thereof having 85%, 8%8, 90%, 91%, 95%, 97%, 98% or 99% identity to SEQ ID NO: 1; and
(b) a beta chain comprising SEQ ID NO: 2 or a functional fragment or derivative thereof having 85%, 8%8, 90%, 91%, 95%, 97%, 98% or 99% identity to SEQ ID NO: 2.

44. The composition according to claim 41, wherein the immune cell is a T cell, a NK T cell or an NK cell.

45. The composition according to claim 41, wherein the cell comprises an antigen recognition moiety that is not the T cell receptor.

46. The composition according to claim 45, wherein the antigen recognition moiety recognition moiety recognizes a tumor antigen.

47. The composition according to claim 41, wherein the cell is autologous to an individual.

48. The composition according to claim 41, wherein the cell is allogeneic to an individual.

49. The composition according to claim 41, wherein the cell expresses a suicide gene product.

50. The composition according to claim 1, wherein the receptor is HLA-A2 restricted.

51. A polynucleotide that expresses an amino acid sequence selected from the group consisting of SEQ ID NO: 1, SEQ ID NO: 2 and SEQ ID NO: 3.

52. An expression vector comprising the polynucleotide according to claim 51.

53. A cell comprising the vector according to claim 52.

54. A method of treating cancer comprising administering to a patient in need thereof a composition according to claim 41.

55. The method according to claim 54, wherein the patient has survivin-positive cancer.

56. The method according to claim 55, wherein the cancer is selected from the group consisting of leukemia, myeloma, breast cancer, lung cancer, colon cancer, melanoma, lymphoma, ovarian cancer, prostate cancer, central nervous system cancer and renal cancer.

57. The method according to claim 54, further comprising providing an additional cancer therapy to the patient.

58. The composition according to claim 44, wherein the immune cell is a T cell.

59. The composition of claim 45, wherein the antigen recognition moiety that is not the T cell receptor is a chimeric antigen receptor or an engager molecule.

60. The composition of claim 46, wherein the tumor antigen is survivin.

61. The composition of claim 49, wherein the suicide gene product is an inducible suicide gene.

62. The composition of claim 61, wherein the inducible suicide gene is iCaspase 9.

63. The method of claim 57, wherein the additional cancer therapy comprises chemotherapy, immunotherapy, radiation, surgery or hormone therapy.


SEQUENCE LISTING (32 sequences)

SEQ ID NO: 1 - protein, 114 aa, Artificial Sequence
  TCR beta chain
DAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRGDSTAEPQHFGDGTRLSIL

SEQ ID NO: 2 - protein, 114 aa, Artificial Sequence
  TCR alpha chain
GESVGLHLPTLSVQEGDNSIINCAYSNSASDYFIWYKQESGKGPQFIIDIRSNMDKRQGQRVTVLLNKTVKHLSLQIAATQPGDSAVYFCAETVTDSWGKLQFGAGTQVVVTPD

SEQ ID NO: 3 - protein, 595 aa, Artificial Sequence
  Synthetic Peptide
MEFGLSWLFLVAILKGVQCDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRGDSTAEPQHFGDGTRLSILEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSRAEGRGSLLTCGDVEENPGPMEFGLSWLFLVAILKGVQCGESVGLHLPTLSVQEGDNSIINCAYSNSASDYFIWYKQESGKGPQFIIDIRSNMDKRQGQRVTVLLNKTVKHLSLQIAATQPGDSAVYFCAETVTDSWGKLQFGAGTQVVVTPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 4 - DNA, 8184 bp, Artificial Sequence
  Synthetic Primer
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggtcaacgcgttagcatgctagcaccggtgccaccatggaatttggcctgagctggctgttcctggtggccatcctgaagggcgtgcagtgcgacgccatggtcatccagaaccccagataccaggtcacacagttcggcaagcccgtgaccctgagctgcagccagaccctgaaccacaacgtgatgtactggtatcagcagaagtccagccaggcccccaagctgctgttccactactacgacaaggacttcaacaacgaggccgacacccccgacaacttccagagcagacggcccaataccagcttctgcttcctggacatcagaagtcctggcctgggcgacgccgccatgtacctgtgtgccaccagcagaggcgacagcaccgccgagcctcagcactttggcgacggcacccggctgagcatcctggaagatctgcggaacgtgaccccccccaaggtgtccctgttcgagcccagcaaggccgagatcgccaacaagcagaaagccaccctcgtgtgcctggccagaggcttcttccccgaccacgtggaactgtcttggtgggtcaacggcaaagaggtgcacagcggcgtcagcaccgacccccaggcctacaaagagagcaactacagctactgcctgagcagccggctgagagtgtccgccaccttctggcacaacccccggaaccacttcagatgccaggtgcagttccacggcctgagcgaagaggacaagtggcccgagggcagccccaagcctgtgacccagaacatcagcgccgaggcctggggcagagccgactgtggcatcaccagcgccagctaccaccagggcgtgctgagcgccaccatcctgtacgagatcctgctgggcaaggccaccctgtacgccgtgctggtgtctggcctggtgctgatggccatggtcaagaagaagaacagcagagccgagggcagaggcagtctgctgacctgcggcgacgtggaagagaaccctggccctatggaatttggactctcctggctgtttctcgtcgctattctgaaaggcgtccagtgtggcgagagcgtgggcctgcatctgcccaccctgtccgtgcaggaaggcgacaacagcatcatcaactgcgcctacagcaacagcgccagcgactacttcatctggtacaagcaggaaagcggcaagggcccccagttcatcatcgacatccggtccaacatggacaagcggcagggccagcgcgtgaccgtgctgctgaacaagaccgtgaagcacctgagcctgcagatcgccgccacccagcctggcgatagcgccgtgtacttctgcgccgagacagtgaccgacagctggggcaagctgcagttcggagccggcacccaggtggtggtcacccccgatatccagaatcccgagcccgccgtgtaccagctgaaggaccccagaagccaggacagcaccctgtgcctgttcaccgacttcgacagccagatcaacgtgcccaagaccatggaatccggcaccttcatcaccgataagaccgtgctggacatgaaggccatggacagcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgcggatcctgctgctgaaggtggccggcttcaacctgctgatgaccctgcggctgtggtccagctgagcatgcacctcgagatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 5 - DNA, 1788 bp, Artificial Sequence
  Synthetic Primer
atggaatttggcctgagctggctgttcctggtggccatcctgaagggcgtgcagtgcgacgccatggtcatccagaaccccagataccaggtcacacagttcggcaagcccgtgaccctgagctgcagccagaccctgaaccacaacgtgatgtactggtatcagcagaagtccagccaggcccccaagctgctgttccactactacgacaaggacttcaacaacgaggccgacacccccgacaacttccagagcagacggcccaataccagcttctgcttcctggacatcagaagtcctggcctgggcgacgccgccatgtacctgtgtgccaccagcagaggcgacagcaccgccgagcctcagcactttggcgacggcacccggctgagcatcctggaagatctgcggaacgtgaccccccccaaggtgtccctgttcgagcccagcaaggccgagatcgccaacaagcagaaagccaccctcgtgtgcctggccagaggcttcttccccgaccacgtggaactgtcttggtgggtcaacggcaaagaggtgcacagcggcgtcagcaccgacccccaggcctacaaagagagcaactacagctactgcctgagcagccggctgagagtgtccgccaccttctggcacaacccccggaaccacttcagatgccaggtgcagttccacggcctgagcgaagaggacaagtggcccgagggcagccccaagcctgtgacccagaacatcagcgccgaggcctggggcagagccgactgtggcatcaccagcgccagctaccaccagggcgtgctgagcgccaccatcctgtacgagatcctgctgggcaaggccaccctgtacgccgtgctggtgtctggcctggtgctgatggccatggtcaagaagaagaacagcagagccgagggcagaggcagtctgctgacctgcggcgacgtggaagagaaccctggccctatggaatttggactctcctggctgtttctcgtcgctattctgaaaggcgtccagtgtggcgagagcgtgggcctgcatctgcccaccctgtccgtgcaggaaggcgacaacagcatcatcaactgcgcctacagcaacagcgccagcgactacttcatctggtacaagcaggaaagcggcaagggcccccagttcatcatcgacatccggtccaacatggacaagcggcagggccagcgcgtgaccgtgctgctgaacaagaccgtgaagcacctgagcctgcagatcgccgccacccagcctggcgatagcgccgtgtacttctgcgccgagacagtgaccgacagctggggcaagctgcagttcggagccggcacccaggtggtggtcacccccgatatccagaatcccgagcccgccgtgtaccagctgaaggaccccagaagccaggacagcaccctgtgcctgttcaccgacttcgacagccagatcaacgtgcccaagaccatggaatccggcaccttcatcaccgataagaccgtgctggacatgaaggccatggacagcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgcggatcctgctgctgaaggtggccggcttcaacctgctgatgaccctgcggctgtggtccagctga

SEQ ID NO: 6 - protein, 11 aa, Artificial Sequence
  Synthetic Peptide
CAARAGNMLTF

SEQ ID NO: 7 - protein, 10 aa, Artificial Sequence
  Synthetic Peptide
CAVNNARLMF

SEQ ID NO: 8 - protein, 14 aa, Artificial Sequence
  Synthetic Peptide
CAMREGGGYNKLIF

SEQ ID NO: 9 - protein, 15 aa, Artificial Sequence
  Synthetic Peptide
CATSRGDSTAEPQHF

SEQ ID NO: 10 - protein, 14 aa, Artificial Sequence
  Synthetic Peptide
CAWGTGLALYEQYF

SEQ ID NO: 11 - protein, 11 aa, Artificial Sequence
  Synthetic Peptide
CAWSIGAEQFF

SEQ ID NO: 12 - protein, 12 aa, Artificial Sequence
  Synthetic Peptide
CAGQDLNTEAFF

SEQ ID NO: 13 - protein, 10 aa, Artificial Sequence
  Synthetic Peptide
  misc_feature (1)..(1): Xaa can be any naturally occurring amino acid
  misc_feature (4)..(4): Xaa can be any naturally occurring amino acid
  misc_feature (10)..(10): Xaa can be any naturally occurring amino acid
XLTXGEFLKX

SEQ ID NO: 14 - protein, 10 aa, Artificial Sequence
  Synthetic Peptide
  misc_feature (1)..(3): Xaa can be any naturally occurring amino acid
  misc_feature (5)..(6): Xaa can be any naturally occurring amino acid
XXXLXXFLKL

SEQ ID NO: 15 - protein, 10 aa, Artificial Sequence
  Synthetic Peptide
ELTLGEFLKL

SEQ ID NO: 16 - protein, 9 aa, Artificial Sequence
  Synthetic Peptide
LMLGEFLKL

SEQ ID NO: 17 - protein, 9 aa, Artificial Sequence
  Synthetic Peptide
NLTHVLYPV

SEQ ID NO: 18 - protein, 9 aa, Artificial Sequence
  Synthetic Peptide
GILGFVFTL

SEQ ID NO: 19 - protein, 14 aa, Artificial Sequence
  Synthetic Peptide
CAETVTDSWGKLQF

SEQ ID NO: 20 - protein, 10 aa, Artificial Sequence
  Synthetic Peptide
ELTLGEFLKL

SEQ ID NO: 21 - protein, 9 aa, Artificial Sequence
  Synthetic Peptide
LALGVFCFA

SEQ ID NO: 22 - protein, 10 aa, Artificial Sequence
  Synthetic Peptide
LLALGVFCFA

SEQ ID NO: 23 - protein, 10 aa, Artificial Sequence
  Synthetic Peptide
QCLLGTFFTC

SEQ ID NO: 24 - protein, 10 aa, Artificial Sequence
  Synthetic Peptide
HIILGLFGLL

SEQ ID NO: 25 - protein, 10 aa, Artificial Sequence
  Synthetic Peptide
NIALGVFALA

SEQ ID NO: 26 - protein, 10 aa, Artificial Sequence
  Synthetic Peptide
QLLLGQFTLL

SEQ ID NO: 27 - protein, 10 aa, Artificial Sequence
  Synthetic Peptide
LLLLGVFAAA

SEQ ID NO: 28 - protein, 10 aa, Artificial Sequence
  Synthetic Peptide
QAYLALFLKL

SEQ ID NO: 29 - protein, 9 aa, Artificial Sequence
  Synthetic Peptide
NLTHVLYPV

SEQ ID NO: 30 - protein, 10 aa, Artificial Sequence
  Synthetic Peptide
ALYVDSLFFL

SEQ ID NO: 31 - protein, 10 aa, Artificial Sequence
  Synthetic Peptide
ELAGIGILTV

SEQ ID NO: 32 - protein, 9 aa, Artificial Sequence
  Synthetic Peptide
YMDGTMSQV